(19) United States
(12) Patent Application Publication
(10) Pub. No.: US 2023/0201124 A1
(43) Pub. Date: Jun. 29, 2023

(54) LUBRICANT FOR PHARMACEUTICALS AND NUTRACEUTICALS

(71) Applicant: JRS Pharma GmbH & Co. KG, Rosenberg (DE)

(72) Inventors: Kenneth L. Seufert, South Kent, CT (US); Anthony Carpanzano, Patterson, NY (US); Theodore Montalto, New Milford, CT (US)

(73) Assignee: JRS Pharma GmbH & Co. KG, Rosenberg (DE)

(21) Appl. No.: 18/089,302

(22) Filed: Dec. 27, 2022

Related U.S. Application Data
(60) Provisional application No. 63/294,540, filed on Dec. 29, 2021.

(51) Int. Cl. A61K 9/20

(52) U.S. Cl. CPC A61K 9/2068

(57) ABSTRACT

Solid dosage forms, tablet processing aids, and methods of preparing solid dosage forms and tablet processing aids are described. The solid dosage forms contain a lubricant contains a rice bran wax. The amount of the rice bran wax in the lubricant is from about 60% to about 100% (w/w).

This application claims priority from U.S. Provisional Application No. 63/294,540, filed Dec. 29, 2021, hereby incorporated by reference in its entirety.

### FIELD OF THE INVENTION

The present invention is related to solid dosage forms comprising a lubricant and methods of preparing solid dosage forms.

### BACKGROUND OF THE INVENTION

Solid dosage forms include powders, granules, tablets and capsules.

Tablets are manufactured through a series of steps comprising a step of compression of a material to be tableted (e.g., a granulate or a mixture of particles) into a shape using a tablet press. A tablet press includes a lower punch which fits into a die from the bottom and an upper punch having a corresponding shape and dimension which enters the die cavity from the top after the material fills the die cavity. The tablet is formed by pressure applied on the lower and upper punches.

A material to be tableted must flow freely into the die to ensure that there is a uniform filling of the die and a continuous movement of the material from the source of the material, e.g., a feeder hopper. The compressed tablet must be readily ejectable from the punch faces of the die without sticking, cracking or breakage. To fulfill these requirements, lubricants are added to the material to be compressed into the tablet.

Commonly used lubricants for preparation of tablets include, e.g., sodium stearyl fumarate (Pruv®), hydrogenated cottonseed oil (Lubritab®) and magnesium stearate. As an alternative to magnesium stearate, natural based lubricants or excipient premixes are sometimes used.

Nu-RICE® (rice extract) is a hypoallergenic extract from rice bran and contains the functional components of the rice bran (lipids, proteins and complex carbohydrates). Fat comprises 15 to 26% of Nu-RICE® by weight.

CompactCel® LUB is a micronized and homogeneous powder compound of hydroxypropylcellulose (HPC), oil, wax and vegetable rice extract (Nu-RICE®).

It was observed that sodium stearyl fumarate (Pruv®), hydrogenated cottonseed oil (Lubritab®) and magnesium stearate upon incorporation into a tablet cause a decrease in a hardness of the tablet.

The decrease may cause the tablet to disintegrate and/or dissolve prematurely and/or too rapidly and not provide the desired effect at the desired time and/or for a desired duration.

A decrease in the tablet hardness may also necessitate a need to include additional amounts of other excipients (e.g., binders) in the tablet, e.g., to prevent the tablet from disintegrating prematurely and too rapidly. Additional amounts of excipients increase the weight and size of the tablet, potentially making it more difficult for the tablet to be swallowed.

### OBJECTS AND SUMMARY OF THE INVENTION

It is an object of the present invention to provide a solid dosage form.

It is an additional object of the present invention to provide a solid dosage form comprising a lubricant that is capable of reducing adhesion of the tablet post compression, minimizing ejection force and punch face adhesion, and does not have a negative impact on the compaction and disintegration/dissolution of the resulting solid dosage form.

It is a further object of the present invention to provide a solid dosage form comprising a lubricant that is a natural material or is derived from a natural material (e.g., a seed of a grass).

It is an additional object of the present invention to provide a solid dosage form comprising a lubricant that is a natural excipient that is primarily comprised from hydrophobic material(s) (i.e., the hydrophobic material(s) comprise(s) 50% or more of the lubricant by weight).

It is an additional object of the present invention to provide a solid dosage form comprising a lubricant consisting essentially of material(s) that are designated by the United States Food and Drug Administration (FDA) as GRAS (Generally Recognized As Safe).

It is a further object of the present invention to provide a solid dosage form comprising a lubricant comprising a rice bran wax.

It is an additional object of the present invention to provide a tablet comprising a lubricant that is a natural material or is derived from a natural material.

It is an additional object of the present invention to provide a tablet comprising a lubricant that is a natural excipient that is primarily comprised from hydrophobic material(s) (i.e., the hydrophobic material(s) comprise(s) 50% or more of the lubricant by weight).

It is an additional object of the present invention to provide a tablet comprising a lubricant that provides a comparable reduction of punch face adhesion as sodium stearyl fumarate (Pruv®), hydrogenated cottonseed oil (Lubritab®) or magnesium stearate, but allows for a lower ejection force.

It is a further object of the present invention to provide a lubricant for use in solid dosage forms.

It is a further object of the present invention to provide a lubricant comprising a rice bran wax.

It is an additional object of the present invention, to provide a method for manufacturing solid dosage forms.

In accordance with the above objects and others which will be obvious to those skilled in the art, the present invention is directed in part to a solid dosage form comprising a tableting aid which includes a lubricant, the lubricant comprising a rice bran wax.

The rice bran wax preferably comprises from 60% to about 100%, about 80% to about 100%, about 90% to about 100%, about 95% to about 100%, or about 98% to about 100% of the lubricant by weight. The tableting aid further comprises one or more additional excipient(s). The lubricant preferably comprises from about 0.1% to about 3% of the tablet by weight. The solid dosage form preferably comprises a drug or a nutraceutical. The solid dosage form is preferably a tablet, but can also be incorporated into a capsule.

The present invention is further directed in other embodiments to a method of preparing the solid dosage form of claim 1, comprising: forming a mixture comprising an active agent, a tableting aid comprising a lubricant, the lubricant comprising a rice bran wax, the rice bran wax comprising from about 60% to about 100% of the lubricant by weight; and compressing the mixture into a tablet.

The present invention is further directed to a pharmaceutical tableting aid comprising rice bran wax, and at least one additional pharmaceutically acceptable excipient selected from the group consisting of an inert diluent, a binder; a disintegrant, a glidant, a flow regulator; and a decomposition accelerator. The rice bran wax and the at least one additional pharmaceutically acceptable excipient are preferably co-processed granules, and are preferably co-processed via a process selected from spray-drying, compaction or granulation. The pharmaceutical tableting aid preferably comprises from about 10% to about 40% of an inert diluent; from about 0% to about 5% of a glidant; and from about 0% to about 10% of one or more additional pharmaceutically acceptable excipients. It may further comprise from about 1% to about 20% of a flow regulator and/or from about 1% to about 20% of a decay accelerator. Most preferably, the pharmaceutical tableting aid includes an inert diluent and/or a binder.

The present invention is further directed to a method of improving the hardness of a compressed solid dosage form, comprising incorporating a lubricant comprising rice bran wax, wherein the rice bran wax comprises from about 0.1% to about 3% of the solid dosage form by weight. Preferably, the rice bran wax is incorporated into a tableting aid which further comprises co-processed granules of the rice bran wax and at least one additional pharmaceutically acceptable excipient selected from the group consisting of an inert diluent, a binder; a disintegrant, a glidant, a flow regulator; and a decomposition accelerator, and the method further comprises co-processing the rice bran wax and the at least one additional pharmaceutically acceptable excipient via a process selected from spray-drying, compaction or granulation, and thereafter mixing the co-processed tableting aid with an effective amount of an active agent. In certain preferred embodiments, the method further comprises compressing the mixture into tablets.

The present invention is further directed to a solid dosage form comprising a lubricant, the lubricant comprising a wax, the wax comprising from about 60% to about 100%, about 85% to about 100%, about 90% to about 100%, about 95% to about 100%, or about 98% to about 100% of the lubricant by weight. The wax could, e.g., be of a type that, upon mixing with a material to be compressed into the solid dosage form and subsequent compression into the solid dosage form, provides a solid dosage form having a hardness greater than a hardness of a solid dosage form compressed from a material that does not comprise the lubricant. The lubricant may provide for a variety of clean label formulations and solid dosage forms containing natural and simple excipients that are easy to recognize, understand and pronounce. The lubricant may also provide for a manufacture of a solid dosage form that is more resisting to cracking and/or breakage as compared to a conventional solid dosage form that does not comprise the lubricant. In some of the embodiments, the lubricant may provide for a manufacture of a solid dosage form having a reduce size and/or weight, as compared to a solid dosage form prepared from a material that does not comprise the lubricant.

The present invention is specifically directed to a solid dosage form comprising a lubricant, the lubricant comprising a rice bran wax, the rice bran wax comprising from 60% to about 100%, about 80% to about 100%, about 90% to about 100%, about 95% to about 100%, or about 98% to about 100% of the lubricant by weight. The rice bran wax may, e.g., comprise (i) aliphatic acids, (ii) alcohol esters and (iii) free fatty acids, the free fatty acids comprising from about 2% to about 7.5% of the rice bran wax by weight. The rice bran wax may also comprise palmitic acid (C16), behenic acid (C22), lignoceric acid (C24), ceryl alcohol (C26), melissyl alcohol (C30), and one or more additional component(s) selected from a group consisting of free fatty acids, squalene and phospholipids. In some of the embodiments, the lubricant may consist of the rice bran wax. In additional embodiments, in addition to the rice bran wax, the lubricant may further comprise one or more additional excipient(s). Generally, the rice bran comprises from about 0.1% to about 5%, about 0.1% to about 3%, or about 0.1% to about 2% of the solid dosage form by weight. In some of the embodiments, the rice bran wax provides an increase in a hardness of the solid dosage form, as compared to the solid dosage form prepared under identical conditions from a material that does not comprise the rice bran wax but is otherwise identical. The invention encompasses solid dosage forms comprising a compressed material comprising the lubricant comprising the rice ban wax. The compressed material may be in a core of the solid dosage form and/or in a coating of the solid dosage form. In some of the preferred embodiments, the solid dosage form is a tablet. The invention also encompasses solid dosage forms comprising a free flowing plurality of particles comprising rice bran wax. In addition to the lubricant comprising the rice bran wax, the solid dosage form may comprise a drug and/or a nutraceutical.

The present invention is also directed to a solid dosage form comprising a lubricant, the lubricant comprising a wax having a melting point from about 77° C. to about 86° C., a saponification value from about 75 to about 120 and a density from about 0.8 to about 1.2 g/cc at 25° C., the wax comprising (i) aliphatic acids, (ii) alcohol esters and (iii) free fatty acids, the free fatty acids comprising from about 2% to about 7.5% of the wax by weight; wherein the wax comprises from about 60% to about 100%, from about 80% to about 100%, about 85% to about 100%, about 90% to about 100%, about 95% to about 100%, or about 98% to about 100% of the lubricant by weight. In some of the embodiments, the density is about 0.96 g/cc at 25° C. and the melting point is 77° C. to about 82° C.

The present invention is also directed to a solid dosage form comprising a lubricant, the lubricant comprising a wax comprising palmitic acid (C16), behenic acid (C22), and lignoceric acid (C24), ceryl alcohol (C26), melissyl alcohol (C30), and one or more additional component(s) selected from a group consisting of free fatty acids, squalene and phospholipids. In the preferred embodiments, the palmitic acid (C16), the behenic acid (C22), the lignoceric acid (C24); the ceryl alcohol (C26), the melissyl alcohol (C30), and the one or more additional component(s) collectively comprise from about 60% to about 100%, from about 80% to about 100%, about 85% to about 100%, about 90% to about 100%, about 95% to about 100%, or about 98% to about 100% of the lubricant by weight.

The present invention is also directed to a solid dosage form comprising a lubricant, the lubricant comprising rice bran wax, the rice bran wax comprising from 60% to about 100%, about 80% to about 100%, about 90% to about 100%, about 95% to about 100%, or about 98% to about 100% of the lubricant by weight.

The present invention is further directed to a solid dosage form comprising a lubricant, the lubricant comprising a wax, the wax comprising from about 60% to about 100% of the lubricant by weight, the wax comprises (i) aliphatic acids, (ii) alcohol esters and (iii) free fatty acids, the free fatty acids comprising from about 2% to about 7.5% of the wax by weight; and the wax has a melting point from about 77° C. to about 86° C., a saponification value from about 75 to about 120 and a density from about 0.8 to about 1.2 g/cc at 25° C. The wax may, e.g., comprise palmitic acid (C16), behenic acid (C22), lignoceric acid (C24), ceryl alcohol (C26), melissyl alcohol (C30), and one or more additional component(s) selected from a group consisting of free fatty acids, squalene and phospholipids. In certain embodiments, the palmitic acid (C16), the behenic acid (C22), the lignoceric acid (C24); the ceryl alcohol (C26), the melissyl alcohol (C30), and the one or more additional component(s) collectively comprise from about 60% to about 100% of the lubricant by weight. In the preferred embodiments, the lubricant comprises rice bran wax. The rice bran wax may, e.g., comprise from 60% to about 100%, about 80% to about 100%, about 90% to about 100%, about 95% to about 100%, or about 98% to about 100% of the lubricant by weight. In some of the embodiments, in addition to the wax, the lubricant is incorporated into a pharmaceutical tableting aid which further comprises one or more additional excipient(s). In the preferred embodiments, the solid dosage form is a tablet (e.g., a compressed tablet) comprising from about 0.1% to about 3% of the lubricant by weight. In addition to the lubricant, the tablet may further comprise one or more drug(s) and/or nutraceutical(s). In other embodiments, the solid dosage form is a capsule containing the (optional) active ingredient (drug) and/or nutraceutical(s) and rice bran wax, along with additional optional pharmaceutically acceptable excipients as described herein. The solid dosage forms and lubricants of the invention may be free from hydroxypropylcellulose (HPC).

In the preferred embodiments, the solid dosage form is a compressed tablet and comprises a lubricant that allows, e.g., for an increase in a hardness of a tablet, as compared to a tablet compressed under identical conditions from a material that does not comprise the lubricant but is otherwise identical to the material to be compressed. Generally, the lubricant of the invention does not adversely affect the compaction and disintegration/dissolution of the resulting tablets.

The present invention is further directed to a solid dosage form comprising an agglomerate comprising the lubricant described herein and an active ingredient(s), one or more optional or additional excipient(s).

The present invention is further directed to a solid dosage form comprising an agglomerate consisting of the lubricant described herein and one or more excipient(s).

The present invention is further directed to a solid dosage form comprising a granulate comprising the lubricant described herein and an active ingredient(s), one or more optional or additional pharmaceutical excipient(s).

The present invention is further directed to a solid dosage form comprising a granulate consisting of the lubricant described herein and one or more optional or additional pharmaceutical excipient(s).

The present invention is further directed to a solid dosage form comprising a powder comprising a mixture comprising the lubricant described herein, an active ingredient(s) and one or more optional or additional pharmaceutical excipient(s).

In addition, the present invention is further directed to a solid dosage form comprising a compressed mixture of an active ingredient(s), a lubricant described herein and one or more optional or additional excipient(s), wherein the active ingredient(s) and one or more optional or additional excipient(s) have been subjected to a wet granulation procedure prior to being incorporated into the mixture with the lubricant.

The present invention is further directed to a solid dosage form comprising the lubricant described herein, the lubricant comprising from about 0.1% to about 5%, from about 0.1 to 3%, from about 0.1 to about 1.5%, or from about 0.1 to about 1% of the solid dosage form by weight. The solid dosage form may consists of the lubricant and one or more additional excipient(s) or may comprise the lubricant, one or more active ingredient(s) and one or more additional excipient(s). The one or more active ingredient(s) may be selected from a group consisting of drugs (i.e., products that are intended for use in the diagnosis, cure, mitigation, treatment, or prevention of disease) and nutraceuticals (e.g., vitamins) and comprise from about 0% to about 99.9% of the solid dosage form by weight. In the preferred embodiments, the solid dosage form is a tablet, and the lubricant is rice bran wax.

The present invention is further directed to a compressed tablet comprising an active ingredient(s) and the lubricant described herein, wherein the active ingredient(s) and the lubricant have been directly compressed into the tablet.

The solid dosage form of the invention may provide a suitable immediate release dissolution profile of the active ingredient(s) when exposed to aqueous solutions during in-vitro dissolution testing, and provides a release of drug in an environment of use which is considered bioavailable. In some of the embodiments of the invention, the dissolution profile of the solid dosage form is modified to provide a controlled or sustained release dissolution profile, e.g., by including an effective amount of a controlled release carrier and/or modifying a compression strength.

The solid dosage form of the invention specifically encompass solid dosage forms for oral administration.

The present invention is also directed to a solid dosage form comprising a lubricant, the lubricant comprising a wax co-processed with one or more additional excipient(s), the wax comprising from about 60% to about 100%, about 85% to about 100%, about 90% to about 100%, about 95% to about 100%, or about 98% to about 100% of the lubricant by weight. In some of the embodiments, the wax is rice bran wax.

The present invention is further directed to methods of preparing solid dosage forms. The solid dosage form may be prepared, e.g., by mixing the lubricant described herein with additional component(s) of a solid dosage form to form a mixture and compressing the mixture into a tablet. The compression into tablets step may be accomplished by any means known to those skilled in the art. In certain embodiments, the ingredients are formulated into compressed tablets by direct compression.

Alternatively, the mixture of the lubricant and other components of the solid dosage form may be incorporated in a granular or powder form into a capsule.

The present invention is directed in part to a method of preparing solid dosage form comprising: forming a mixture comprising a lubricant, the lubricant comprising a rice bran wax, the rice bran wax comprising from about 60% to about 100% of the lubricant by weight, and compressing the mixture into a tablet.

The present invention is directed in part to a method of preparing a solid dosage form comprising: forming a granulation comprising a lubricant, the lubricant comprising a rice bran wax, the rice bran wax comprising from about 60% to about 100% of the lubricant by weight, and compressing the granulation into a tablet.

The present invention is directed in part to a method of preparing a solid dosage form comprising: forming a mixture comprising (i) a lubricant comprising a rice bran wax, the rice bran wax comprising from about 60% to about 100% of the lubricant by weight, and (ii) an active ingredient, and compressing the mixture into a tablet.

### Definitions

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

The terms “a” and “an” do not denote a limitation of quantity, but rather denote the presence of at least one of the referenced item.

The term “about” is used synonymously with the term “approximately.” The use of the term “about” with respect to doses and amounts indicates that values slightly outside the cited values, i.e., plus or minus 0.1% to 20%.

An “active agent” is any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. The terms “active agent” and “drug” and “active ingredient” are used synonymously herein and include drugs and nutraceuticals.

A “drug” for the purposes of the invention means a products that is intended for use in the diagnosis, cure, mitigation, treatment, or prevention of a disease.

By “controlled release” or “sustained release” it is meant for purposes of the invention that the therapeutically active medicament is released from the formulation at a controlled rate such that therapeutically beneficial blood levels (but below toxic levels) of the medicament are maintained over an extended period of time, e.g., providing a 12 hour or a 24 hour dosage form.

By “bioavailable” it is meant for purposes of the invention that the therapeutically active medicament is absorbed from the formulation and becomes available in the body at the intended site of drug action.

“Pharmaceutical compositions” are compositions comprising at least one active agent (drug), such as a compound or salt, solvate, or hydrate of an active agent, and at least one other substance, such as a carrier. Pharmaceutical compositions optionally contain one or more additional active agent(s). When specified, pharmaceutical compositions meet the U.S. FDA's GMP (good manufacturing practice) standards for human or non-human drugs. “Pharmaceutical combinations” are combinations of at least two active agents which may be combined in a single dosage form.

“Pharmaceutically acceptable” refers to those properties and/or substances that are acceptable to a human patient from a pharmacological/toxicological point of view and to the manufacturing pharmaceutical chemist from a physical/chemical point of view regarding composition, formulation; stability, patient acceptance and bioavailability.

The term “lubricant” for purposes of the invention means a component for or incorporated into a solid dosage form (i.e., an excipient of a solid dosage form). Being “a component for or incorporated into a solid dosage form” for purposes of the present invention is not a mere intended use of the lubricant. Rather, it is a limitation on the composition, structure and/or grade of the lubricant. For example, the composition, structure and/or grade of the lubricant must be such that it is suitable for oral compatible with other components of the solid dosage form and approved by a health regulatory agency (e.g., U.S. FDA) for administration to humans and/or animals.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“MgSt” is an abbreviation for magnesium stearate.

### BRIEF DESCRIPTION OF THE DRAWINGS

The following drawings are illustrative of embodiments of the invention and are not meant to limit the scope of the invention as encompassed by the claims.

FIG. 1 is a plot of formulations containing each of the lubricants set forth in Table 1 showing tablet harness (N) at various compression forces [kN];

FIG. 2 is a plot of formulations containing each of the lubricants set forth in Table 1 showing ejection force [N] versus tablet harness (N);

FIG. 3 is a plot of formulations containing each of the lubricants set forth in Table 1 showing disintegration time (H:MM:SS) versus tablet crushing strength [N];

FIG. 4 is a plot of formulations containing each of the lubricants set forth in Table 2 showing tablet hardness [N] versus compression force (kN);

FIG. 5 is a plot of each of the lubricants set forth in Table 2 showing ejection force [N] versus tablet harness (N);

FIG. 6 is a plot of ejection force [N] versus tablet hardness [N] for plots containing various amount of MCC and DCP with rice bran wax for formulations of Example 3 containing various amount of MCC and DCP with rice bran wax;

FIG. 7 is another plot of ejection force [N] versus tablet hardness [N] for formulations of Example 3 containing various amount of MCC and DCP with rice bran wax;

FIG. 8 is a plot of tablet hardness [N] versus compression force [kN] for formulations of Example 4 containing various amount of rice bran wax with 50% MCC:50% DCP;

FIG. 9 is a plot showing ejection force [N] versus tablet hardness [N] for formulations of Example 4 containing 50% MCC:50% DCP with various amounts of rice bran wax (0.1, 0.25, 0.5 and 1%);

FIG. 10 is a plot showing tablet hardness [N] versus compression force [kN] for formulations of Example 5 containing 250 mg quercetin dihydrate and 1% rice bran wax with 50% MCC:50% DCP;

FIG. 11 is a plot showing ejection force (N) versus tablet hardness (N) for formulations of Example 5 containing 250 mg quercetin dihydrate and 1% rice bran wax with 50% MCC:50% DCP and 1% glidant (Formulation #3);

FIG. 12 is a plot of turmeric formulations with lubricant of Example 6 showing tablet hardness [N] versus compression force [kN];

FIG. 13 is a plot of turmeric formulations of Example 6 showing ejection force (N) versus tablet hardness (N);

FIG. 14 is a plot of ascorbic acid formulations of Example 7 showing tablet hardness (N) versus compression force (N);

FIG. 15 is a plot of ascorbic acid formulations of Example 7 showing ejection force (N) versus tablet hardness (N);

FIG. 16 is a plot that shows the tablet hardness [N] versus the compression force [kN] for the tablets of Example 8, wherein the lubricant is 0.125% rice bran wax (5 minute blend);

FIG. 17 is a plot that shows the tablet hardness [N] versus compression force [kN] for the tablets of Example 8 wherein 1% lubricant (rice brain wax, sodium stearyl fumarate, magnesium stearate, hydrogenated vegetable oil; 5 minute blend) was used;

FIG. 18 is a plot that shows the tablet hardness [N] versus compression force [kN] for the tablets of Example 8 wherein 1% lubricant (rice brain wax, sodium stearyl fumarate, magnesium stearate, hydrogenated vegetable oil; 60 minute blend) was used;

FIG. 19 is a graph of Example 8 formulations which provides tablet hardness changes with lubricant level and blend time (0.125% rice bran wax for 5 minutes; 1.0% rice bran wax for 60 minutes; other lubricants; 15 kN compaction);

FIG. 20 is a graph of Example 8 formulations which shows ejection force values for each lubricant level and each blending time;

FIG. 21 is a plot of Example 8 formulations showing ejection forces after 60 minutes blending for 1% lubricant (rice bran wax, sodium stearyl fumarate, magnesium stearate, hydrogenated vegetable oil);

FIG. 22 is an NIR spectra of the study components of Example 9;

FIG. 23 is a plot showing the NIR spectra for the aspirin-magnesium stearate blend of Example 9 initially (solid), and after 2 months under different storage conditions; and

FIG. 24 is a plot showing the NIR spectra for the aspirin-magnesium stearate blend of Example 9 initially (solid), and after 2 months under different storage conditions.

### DETAILED DESCRIPTION OF THE INVENTION

The present invention is directed in part to a solid dosage form (e.g., a tablet) comprising a lubricant.

The main functions of a lubricant in a preparation of a tablet are to reduce adhesion of the tablet post compression, reduce the friction between the compressed tablet and the die wall; avoid the material(s) being tableted from sticking to the punches; and minimize ejection force and punch face adhesion. Lubricants further allow, e.g., for the tablet to be cleanly ejected and without cracking or breakage.

The present invention encompasses lubricants that can be used as excipients of solid dosage forms. The lubricant in accordance with the invention is free-flowing and, in some of the preferred embodiments, is directly compressible.

The average particle size of the lubricant of the present invention ranges from about 1 microns to about 1000 microns. Particle sizes of about 2-500 microns are preferred, particle sizes of about 3-250 microns are more preferred and particle sizes of about 4-100 microns are most preferred. In some of these embodiments, a mean particle size of the lubricant is from about 4 m to about 15 m, about 5 m to about 12 m, or about 6 m to about 10 m. In some of these embodiments, the maximum particle size is about 31 m.

The lubricant of the present invention preferably has a bulk (loose) density ranging from about 0.2 g/ml to about 0.6 g/ml, and most preferably from about 0.35 g/ml to about 0.55 g/ml. The lubricant preferably has a tapped density ranging from about 0.2 g/ml to about 0.6 g/ml, and most preferably from about 0.35 g/ml to about 0.55 g/ml. The pH of the particles is most preferably about neutral, although granulates having a pH of from about 3.0 to about 8.5 are possible. The moisture content of the excipient particles will preferably broadly range from about 0.5% to about 15%, preferably from about 2.5% to about 6%, and most preferably from about 3.0% to about 5% by weight.

The lubricant may be mixed in the desired proportion with an active agent and, if desired, with one or more additional excipients (blended or dry granulated), and then directly compressed into a solid dosage form (e.g., a tablet).

The lubricant of the invention allows, e.g., for the compressed tablet to be ejected from the die with an ejection force of 500 N or less, 250 N or less, 200 N or less or 150 N or less, and the compressed tablet having a hardness from about 20 N to about 80 N, from about 40 N to about 80 N or from about 45N to about 75N.

In certain embodiments of the present invention, the lubricant may be co-processed with one or more additional excipients, prior to incorporation into a solid dosage form.

In certain embodiments of the present invention, the lubricant may be co-processed with microcrystalline cellulose, prior to incorporation into a solid dosage form.

In certain embodiments of the present invention, the lubricant and an active ingredient may be co-processed with one or more controlled or sustained release carriers to provide a delayed or sustained release of the active ingredient from the final product (e.g., oral tablet). This can be accomplished, e.g., by incorporating a sustained release carrier(s) together with the mixture of the lubricant and a drug(s) (with further optional drugs and or further optional pharmaceutically acceptable excipients) and then tableting the mixture, thereby obtained sustained release matrix tablets.

In addition, the lubricant and the drug(s) (with further optional drugs and or further optional pharmaceutically acceptable excipients) may be tableted or filled into a capsule, which then may be coated with one or more delayed (e.g., enteric) or sustained release carriers to thereby provide a delayed or sustained release final formulation.

### Rice Bran Wax

In certain embodiments of the invention, the solid dosage form of the invention comprises a lubricant, and the lubricant comprises rice bran wax.

Rice bran wax is a hard, crystalline, high melting vegetable wax obtained from rice (Oryza Sativa). The main components of rice bran wax are aliphatic acids (wax acids) and higher alcohol esters including, e.g., long chain saturated C₄₆-C₆₂ esters from C₂₀-C₃₆ fatty alcohols and C₂₀-C₂₆ fatty acids. The higher alcohol esters include, e.g., ceryl alcohol (C₂₆) and melissyl alcohol (C₃₀). The aliphatic acids contained in rice bran wax include, e.g., palmitic acid (C₁₆), behenic acid (C₂₂), and lignoceric acid (C₂₄). Rice bran wax also contains constituents such as free fatty acids (e.g., palmitic acid), squalene and phospholipids. Free fatty acids generally comprise from about 2.1 to about 7.3% of the rice bran wax by weight.

In some of the preferred embodiments of the invention, rice bran wax is micronized and has a melting point from about 77° C. to about 82° C.

In some of the embodiments, the rice bran wax is Naturefine® R331, which has been treated to and/or co-processed with an additional excipient to render Naturefine® R331 suitable for incorporation into a solid dosage form and/or improve it lubricating properties.

Contrary to what is typical of lubricants, tablets lubricated with rice bran wax showed an increase in tablet hardness with increasing compression forces and blending times. This suggests that rice bran wax is acting as a binder to some degree (without negatively impacting ejection forces).

### Solid Dosage forms

Solid dosage forms of the invention encompass solid dosage forms consisting of excipients (i.e., “placebo” dosage forms), solid dosage forms comprising drugs, and solid dosage forms comprising nutraceuticals.

Solid dosage forms of the invention include powders, granules, tablets, and capsules.

Solid dosage forms of the invention comprise a lubricant. The lubricant comprises a wax. Generally, the wax comprises from about 60% to about 100% of the lubricant by weight. In the preferred embodiments, the wax is rice bran wax.

In some of the preferred embodiments of the invention, the lubricant is uniformly dispersed throughout a solid dosage form.

In other preferred embodiments, the lubricant of the invention is uniformly dispersed throughout a core of the solid dosage.

In additional preferred embodiments, the solid dosage form comprises a compressed coating, and the lubricant is uniformly dispersed throughout the compressed coating.

### Solid Dosage Forms

In the preferred embodiments of the invention, the solid dosage form is a tablet or a capsule which comprises a lubricant. The lubricant comprises a wax. Generally, the wax comprises from about 60% to about 100% of the lubricant by weight. In the preferred embodiments, the wax is rice bran wax.

In certain preferred embodiments, the invention is directed to a tablet comprising from about 0.1 to about 5.0%, more preferably from about 0.125% to about 2.0% of a lubricant comprising or consisting of rice bran wax, from about 1% to about 99.75%, and more preferably from about 10% to about 95% of an inert diluent, from about 0% to about 5% and more preferably from about 0% to about 2% of a glidant, from about 1% to about 90% of an active agent, and from about 0% to about 10% of one or more additional pharmaceutically acceptable excipients.

The average tablet size for round tablets is preferably about 50 mg to 500 mg and for capsule-shaped tablets about 200 mg to 2000 mg. However, other formulations prepared in accordance with the present invention may be suitably shaped for other uses or locations, such as other body cavities, e.g., periodontal pockets, surgical wounds, or vaginally. It is contemplated that for certain uses, e.g., antacid tablets, vaginal tablets and possibly implants, that the tablet will be larger.

In certain embodiments, the tablets are directly compressible tablets.

In certain embodiments, the tablet is a compression coated tablet, in which the active substance is contained within a core which is contained within an outer coating (either hydrophobic coating or hydrophilic coating, e.g., as described below). In some embodiments, the coating may be complete, in other embodiments, the coating may be partial.

The solid dosage forms prepared using the novel lubricant may also include suitable quantities of pharmaceutical adjuvants, e.g., diluents, plasticizers, binders, granulating aids, disintegrants (e.g., sodium starch glycolate (commercially available from JRS Pharma under the tradename Explotab©), colorants, flavorants and glidants that are conventional in the pharmaceutical art.

Binders are substances which are added to promote cohesive compacts for directly compressed tablets, A non-limiting list of binders include microcrystalline cellulose, starches, lactose, sugar alcohols (e.g., mannitol, sorbitol, and the like), polymers (e.g., PVP, PEG, HPC, HPMC, Methylcellulose, polymethacrylates, sodium carboxy methyl cellulose, and the like), saccharides and their derivatives (e.g., lactose, sucrose) and isomalt.

A non-limiting list of suitable adjuvants include spray dried lactose, polyvinylpyrrolidone (PVP), alginates (e.g., commercially available from JRS Pharma under the tradename VIVAPHARM®), talc, magnesium stearate (e.g., commercially available from JRS pharma as Lubri-Prez™), and mixtures thereof. The quantities of these additional materials will be sufficient to provide the desired effect to the desired formulation. Other examples of pharmaceutically acceptable carriers and excipients that may be used to formulate oral dosage forms are described in the Handbook of Pharmaceutical Excipients, American Pharmaceutical Association (1986), herein incorporated by reference in its entirety.

A non-limiting list of plasticizers includes include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used.

For example, if necessary, any generally accepted soluble or insoluble inert pharmaceutical filler (diluent) material can be included in the final product (e.g., a tablet). Preferably, the inert pharmaceutical filler comprises a monosaccharide, a disaccharide, a polyhydric alcohol, inorganic phosphates, sulfates or carbonates, and/or mixtures thereof. Examples of suitable inert pharmaceutical fillers include sucrose, dextrose, lactose, xylitol, fructose, sorbitol, calcium phosphate, calcium sulfate, calcium carbonate, “off-the-shelf” microcrystalline cellulose, silicified microcrystalline cellulose, mixtures thereof, and the like.

The tablets of the present invention may also contain effective amounts of coloring agents, (e.g., titanium dioxide, F.D. & C. and D. & C. dyes; see the Kirk-Othmer Encyclopedia of Chemical Technology, Vol. 5, pp. 857-884, herein incorporated by reference), stabilizers, binders, odor controlling agents, and preservatives.

Additional excipients which may be used in the dosage form of the present invention are described in Handbook of Pharmaceutical Excipients, 2^{nd} Edition, American Pharmaceutical Association; The Theory and Practice of Industrial Pharmacy, 2^{nd} Edition, Lachman, Leon, 1976; Pharmaceutical Dosage Forms: Tablets Volume 1, 2^{nd} Edition, Lieberman, Hebert A., et al, 1989; Modern Pharmaceutics, Banker, Gilbert and Rhodes, Christopher T, 1979; and Remington's Pharmaceutical Sciences, 15th Edition, 1975, each herein incorporated by reference.

### Tableting Aid

In certain embodiments, the invention is directed to a tableting aid comprising rice bran wax.

In certain preferred embodiments, the invention is directed to a mixture of a lubricant comprising or consisting of rice bran wax and an inert filler.

In certain embodiments, the invention relates to a process for producing a tableting aid, which comprises the following process steps:—comprising a lubricant (rice bran wax) and at least one of the following components: a filler/binder; a lubricant; a flow regulator; a decomposition accelerator; a disintegrant; a glidant; wherein the components provided are mixed with one another.

Direct compression is the most commonly used method for tablet manufacturing. The procedure consists of different mixing steps. First, the drug and all excipients (mainly filler, binder, and disintegrant) can be mixed together except for the lubricant. This can be done in a single mixing, wherein the lubricant, and optional filler, binder, disintegrant are added together. On the other hand, several consecutive mixing steps can be made. The lubricant is then added to the mixture and mixed again with it. The result is a tableting mixture. This mixture can be pressed on a tablet press.

In pharmaceutical technology, there are combined excipients (direct tableting excipients). These are combined tableting excipients which have been prepared from several individual substances (very frequently fillers, binders and disintegrating agents) by co-processing (for example spray-drying, compaction or granulation).

In certain preferred embodiments, the invention is directed to a tablet comprising from about 0.1 to about 5.0%, more preferably from about 0.125% to about 100.0% (in embodiments where only the lubricant (rice bran wax) is included as the tableting aid) of a lubricant comprising or consisting of rice bran wax; from about 1% to about 99.75%, and more preferably from about 10% to about 95% of an inert diluent; from about 0% to about 5% and more preferably from about 0% to about 2% of a glidant; and from about 0% to about 10% of one or more additional pharmaceutically acceptable excipients.

In certain preferred embodiments, the tableting aid comprises a filler/binder (e.g., microcrystalline cellulose), a lubricant comprising or consisting of rice bran wax, a flow adjusting agent (e.g., from about 1 to about 20% silica, calcium, magnesium and aluminum silicates), The decay accelerator may comprise from about 1 to about 20% croscarmellose sodium, starch, sodium carboxymethyl starch, cross-linked polyvinylpyrrolidone, soy polysaccharide, cyclodextrin, xylan, pectins, gelatin, polymethacrylic acid, ion exchange resins.

The tableting aid of the present invention may provide the following advantages: low ejection forces after compression of the tableting mixture, good lubricating properties and good flow properties of the tableting mixture, sufficient tablet hardness, low abrasion of the tablet, low sensitivity to moisture.

The tableting aid according to the invention preferably contains a plurality of individual substances, generally one or more of a filler, a binder, a disintegrant, a glidant in addition to the lubricant (rice bran wax). All of the substances mentioned are thus present in a single substance mixture; this can therefore be described as multifunctional. This has the great advantage that the user, thus the tablet manufacturer, only needs to mix a finished mixture of individual tableting excipients with the active ingredient of the tablet. It thus eliminates the addition of individual tablet excipients, which labor is saved and the problem of inaccuracies of dosing of the individual tablet excipients does not occur. The invention leads to the following further advantages: significantly lower ejection forces, thanks to the even distribution of the lubricant in the tablet and thus a higher amount of tableting excipients or faster production, better abrasion (especially for low-dose drugs),

In certain preferred embodiments, lower concentrations of lubricant in the tablet are sufficient, an in preferred embodiments, a better tablet hardness is achieved, and/or better flow properties of the tableting compound, and/or a lower moisture sensitivity, and/or reduced or no impact on disintegration and drug release, and/or no overmixing or lids, and/or lower dust count.

Only one mixing step (of the ingredients of the tableting aid) with the active ingredient (s) is required. This saves production time and costs.

The preparation of the mixtures of the abovementioned components is preferably carried out by wet granulation by all known methods such as mixer granulation, perforated disc granulation, fluidized bed granulation, extrusion or shugi granulation.

In the case of fluidized bed granulation, for example, there are the following options:
Variant A: Silicified microcrystalline cellulose and croscarmellose or sodium carboxymethyl starch in a fluidized bed and spray at 30-97° C. with a hot, aqueous solution of a solution or suspension of lubricant (e.g., aqueous suspension of rice bran wax). The granules are dried in the fluidized bed.
Variant B: Introduce microcrystalline cellulose and croscarmellose or sodium carboxymethyl starch in the fluidised bed and spray at 30-97° C. with a hot, aqueous suspension of rice bran wax and silica. The granules are dried in the fluidized bed.
Variant C: Prepare microcrystalline cellulose, croscarmellose or sodium carboxymethyl starch and silica in the fluidised bed and spray at 30-97° C. with a hot, aqueous suspension of rice bran wax. The granules are dried in the fluidized bed.
Variant D: Introduce silicified microcrystalline cellulose in the fluid bed and at 30-97° C. with a hot, aqueous suspension of rice bran wax and croscarmellose or sodium. Spray carboxymethyl starch. The granules are dried in the fluidized bed.

Variant E: Substrate microcrystalline cellulose in the fluidized bed and at 30-97° C. sprayed with a hot, aqueous solution/suspension of tablet excipients, croscarmellose or sodium carboxymethyl starch and silicon dioxide. The granules are dried in the fluidized bed.

### Active Ingredients

The present invention is useful with any active ingredient capable of being formulated as a crystalline or an amorphous drug. The term “drug” is conventional, denoting a compound having beneficial prophylactic and/or therapeutic properties when administered to a human or an animal.

The active ingredient(s) may further be any agent that is traditionally used as a medicament and lends itself to being administered through the oral cavity. Such active agents may be, for example, vitamins, chemotherapeutics; antimycotics; oral contraceptives, nicotine or nicotine replacement agents, minerals, analgesics, antacids, muscle relaxants, antihistamines, decongestants, anesthetics, antitussives, diuretics, anti-inflammatories, antibiotics, antivirals, psychotherapeutic agents, anti-diabetic agents and cardiovascular agents, nutraceuticals and nutritional supplements. This list is exemplary only, and is not meant to be limiting in any way.

Classes of drugs which may be incorporated with the lubricant of the present invention include, but are not limited to, analgesics, anesthetics, antihypertensives, antianxiety agents, anticlotting agents, anticonvulsants, blood glucose-lowering agents, coronary drugs, decongestants, antihistamines, antitussives, diuretics, antineoplastics, beta blockers, anti-inflammatories, antipsychotic agents, cognitive enhancers, anti-atherosclerotic agents, cholesterol-reducing agents, antiobesity agents, autoimmune disorder agents, anti-impotence agents, antibacterial, antiviral and antifungal agents, hypnotic agents, anti-Parkinsonism agents, anti-Alzheimer's disease agents, antibiotics, anti-depressants, antiviral agents, glycogen phosphorylase inhibitors, anti-neoplastic agents, anxiolytics, sedatives and hypnotics, local anesthetics, migraine relieving agents, drugs for treating motion sickness, anti-emetics, chemotherapeutics, cholesterol ester transfer protein inhibitors, botanical bioactive agents, vitamins and co-enzymes, and neutraceuticals.

Coronary drugs include but are not limited to vasodilators such as nitroglycerin, isosorbide dinitrate, Calcium-antagonists such as verapamil, nifedipine and diltiazem, Cardiac-glycosides such as digoxin.

Analgesics include but are not limited to opioid analgesics such as morphine, buprenorphine, oxycodone, oxymorphone, hydromorphone, meperidine, fentanyl, sufentranil, alfentanil, aspirin, acetaminophen, etc; NSAIDs such as naproxen, ibuprofen, diclofenac; Local anesthetics such as lidocaine, bupivacaine, etc.; ergot and ergot derivatives (wigraine, cafergot, ergostat, ergomar, dihydroergotamine), imitrex.

Example of cholesterol and triglycerides lowering drug: fenofibrate, lovastatin, simvastatin, pravastatin, fluvastatin, atorvastatin, or cerivastatin.

Anxiolytics, sedatives and hypnotics include but are not limited to diazepam, nitrazepam, flurazepam, estazolam, flunitrazepam, triazolam, alprazolam, midazolam, temazepam, lormetazepam, brotizolam, clobazam, clonazepam, lorazepam, oxazepam, buspirone, etc;

Migraine relieving agents include but are not limited to sumatriptan, ergotamines and derivatives etc.

Drugs against motion sickness include but are not limited to cinnarizine, antihistamines, etc.

Anti-emetics include but are not limited to ondansetron, tropisetron, granisetrone, metoclopramide, etc. Others: such as disulfuram, vitamin K, etc.

Examples of chemotherapeutics agents include but are not limited to cisplatin (CDDP), procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof.

Antibiotics drugs include but are not limited to tetracyclines such as tetracycline, doxycycline, oxytetracycline, chloramphenicol etc.; macrolides such as erythromycin and derivatives, etc.

Antivirals include but are not limited to acyclovir, idoxuridine, tromantadine etc.;

Antimycotics include but are not limited to miconazole, ketoconazole, fluconazole, itraconazole, econazole, terconazole, griseofulvin, and polyenes such as amphotericin B or nystatine etc.

Anti-amoebics include but are not limited to metronidazole, metronidazole benzoate and tinidazole etc.

Anti-inflammatory drugs include but are not limited to steroids or NSAID's such as indomethacin, ibuprofen, piroxicam, diclofenac etc.; Anti-allergics: Disodium cromoglycate etc.; Immunosuppressive agents: cyclosporins etc.

Antimicrobial agents that may be used include but are not limited to naficillin, oxacillin, vancomycin, clindamycin, erythromycin, trimethoprim-sulphamethoxazole, rifampin, ciprofloxacin, broad spectrum penicillin, amoxicillin, gentamicin, ceftriazoxone, cefotaxime, chloramphenicol, clavunate, sulbactam, probenecid, doxycycline, spectinomycin, cefixime, penicillin G, minocycline, β-lactamase inhibitors; meziocillin, piperacillin, aztreonam, norfloxacin, trimethoprim, ceftazidime, ceftriaxone and dapsone.

Antifungal agents that may be delivered include but are not limited to ketoconazole, fluconazole, nystatin, itraconazole, clomitrazole, and amphotericin B. Antiviral agents that may be used include but are not limited to acyclovir, trifluridine, idoxorudine, foscarnet, ganciclovir, zidovudine, dideoxycytosine, dideoxyinosine, stavudine, famciclovir, didanosine, zalcitabine, rifimantadine, and cytokines.

Antihistamines are represented by but are not limited to cimetidine, ranitidine, diphenydramine, prylamine, promethazine, chlorpheniramine, chlorcyclizine, terfenadine, carbinoxamine maleate, clemastine fumarate, diphenhydramine hydrochloride, dimenhydrinate, prilamine maleate, tripelennamine hydrochloride, tripelennamine citrate, chlorpheniramine maleate, brompheniramine maleate, hydroxyzine pamoate, hydroxyzine hydrochloride, cyclizine lactate, cyclizine hydrochloride, meclizine hydrochloride, acrivastine, cetirizine hydrochloride, astemizole, levocabastine hydrochloride, and loratadine.

Decongestants and antitussives include but are not limited to agents such as dextromethorphan, levopropoxyphene napsylate, noscapine, carbetapentane, caramiphen, chlophedianol, pseudoephedrine hydrochloride, diphenhydramine, glaucine, pholcodine, and benzonatate.

Anesthetics include but are not limited to etomidate, ketamine, propofol, and benodiazapines (e.g., chlordiazepoxide, diazepam, clorezepate, halazepam, flurazepam, quazepam, estazolam, triazolam, alprozolm, midazolam, temazepam, oxazepam, lorazepam), benzocaine, dyclonine, bupivacaine, etidocaine, lidocaine, mepivacaine, promoxine, prilocalne, procaine, proparcaine, ropivacaine, tetracaine. Other useful agents may include amobartital, aprobarbital, butabarbital, butalbital mephobarbital, methohexital, pentobarbital, phenobarbital, secobarbital, thiopental, paral, chloral hydrate, ethchlorvynol, clutethimide, methprylon, ethinamate, and meprobamate.

Diuretics include but are not limited to acetazolamide, dichlorphenamide, methazolamide, furosemide, bumetanide, ethacrynic acid torseimde, azosemide, muzolimine, piretanide, tripamide, bendroflumethiazide, benzthiazide, chlorothiazide, hydrochlorothiazide, hydroflumethiazide, methyclothiazide, polythiazide, trichlormethiazide, indapamide, metolazone, quinethazone, amiloride, triamterene, sprionolactone, canrenone, and potassium canrenoate.

Anti-inflammatories include but are not limited to salicylic acid derivatives (e.g. aspirin) paraminophenol derivative (e.g. acetaminophen) indole and indene acetic acids (indomethacin, sulindac and etodalac) heteroaryl acetic acids (tolmetin diclofenac and ketorolac) aryl propionic acid derivatives (ibuprofen, naproxen, ketoprofen, fenopren, oxaprozine), anthranilic acids (mefenamic acid, meclofenamic acid) enolic acids (piroxicam, tenoxicam, phenylbutazone and oxyphenthatrazone).

Psychotherapeutic agents include but are not limited to thorazine, serentil, mellaril, millazine, tindal, permitil, prolixin, trilafon, stelazine, suprazine, taractan, navan, clozaril, haldol, halperon, loxitane, moban, orap, risperdal, alprazolam, chlordiaepoxide, clonezepam, clorezepate, diazepam, halazepam, lorazepam, oxazepam, prazepam, buspirone, elvavil, anafranil, adapin, sinequan, tofranil, surmontil, asendin, norpramin, pertofrane, ludiomil, pamelor, vivactil, prozac, luvox, paxil, zoloft, effexor, wellbutrin, serzone, desyrel, nardil, parnate, eldepryl.

Cardiovascular agents include but are not limited to nitroglycerin, isosorbide dinitrate, sodium nitroprisside, captopril, enalapril, enalaprilat, quinapril, lisinopril, ramipril, losartan, aminone, lirinone, vesnerinone, hydralazine, nicorandil, prozasin, doxazosin, bunazosin, tamulosin, yohimbine, propanolol, metoprolol, nadolol, atenolol, timolol, esmolol, pindolol, acebutolol, labetalol, phentolamine, carvedilol, bucindolol, verapamil, nifedipine, amlodipine and dobutamine.

Anti-neoplastic agents and immunosuppressants include but are not limited to aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone.

The therapeutic agent(s) which are incorporated into the formulations of the present invention can be used, for example, in the amounts indicated in the Physician's Desk Reference, or as otherwise known and used by one of ordinary skill in the art.

Vitamins and co-enzymes that may be delivered using this invention include but are not limited to water or fat soluble vitamins such as thiamin, riboflavin, nicotinic acid, pyridoxine, pantothenic acid, biotin, flavin, choline, inositol and paraminobenzoic acid, carnitine, vitamin C, vitamin D and its analogs, vitamin A and the carotenoids, retinoic acid, vitamin E and vitamin K and Coenzyme Q10.

Example of botanical bioactive agents, are: polyphenols, isoflavones, resveratrol, soy isoflavones, grape seed extract polyphenols, curcumin, epigenin. Anti-inflammatory plant extracts such as aloe vera, echinacea and chamomile hammamelis extracts, anti-psoriatic such as chinese zizipus jujuba. Astringents such as hammamelis anti-bacterial such as artemisia, chamomile, golden seal. Immune modulators such as echinacea, anti-aging or anti-cancer or anti-photo damage, anti-inflammatory such as feverfew parthenolides, rejuvenation agents, carotenoids, beta-carotene, lycopene, astaxanthons, lutein, tocopheryl and retinol.

In certain embodiments of the invention, the tablet is coated with a sufficient amount of a hydrophobic polymer to render the formulation capable of providing a release of the active ingredient(s) such that a 12 or 24 hour formulation is obtained. The hydrophobic polymer which included in the tablet coating may be the same or different material as compared to the hydrophobic polymeric material which is optionally granulated with the sustained release excipient.

In other embodiments of the present invention, the tablet coating may comprise an enteric coating material in addition to or instead or the hydrophobic polymer coating. Examples of suitable enteric polymers include cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit®L 100-555.

In further embodiments, the dosage form may be coated with a hydrophilic coating in addition to or instead of the above-mentioned coatings. An example of a suitable material which may be used for such a hydrophilic coating is hydroxypropylmethylcellulose (e.g., Opadry®, commercially available from Colorcon, West Point, Pa.).

The coatings may be applied in any pharmaceutically acceptable manner known to those skilled in the art. For example, in one embodiment, the coating is applied via a fluidized bed or in a coating pan. For example, the coated tablets may be dried, e.g., at about 60-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

The coatings which may be optionally applied to the compressed solid dosage form of the invention may comprise from about 0.5% to about 30% by weight of the final solid dosage form.

In additional embodiments of the present invention, a support platform may be applied to the tablets manufactured in accordance with the present invention. Suitable support platforms are well known to those skilled in the art. An example of suitable support platforms is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 microns if applied via spray-coating or immersion-coating. Generally, in embodiments of the invention wherein a hydrophobic polymer or enteric coating is applied to the tablets, the tablets are coated to a weight gain from about 1% to about 20%, and in certain embodiments preferably from about 5% to about 10%.

Materials useful in the hydrophobic coatings and support platforms of the present invention include derivatives of acrylic acid (such as esters of acrylic acid, methacrylic acid, and copolymers thereof) celluloses and derivatives thereof (such as ethylcellulose), polyvinylalcohols, rice bran wax, and the like.

In other embodiments of the invention which provide a sustained release product, the sustained-release carrier may be incorporated in a sustained-release matrix to impart sustained-release of the active agent from the final formulation. The sustained release carrier may be hydrophobic or hydrophilic. Suitable materials which may be included in the sustained release carrier of the present invention include alkylcelluloses such as natural or synthetic celluloses derivatives (e.g. ethylcellulose), acrylic and methacrylic acid polymers and copolymers, zein, rice bran wax, and mixtures thereof. Suitable biocompatible, preferably biodegradable polymers can be utilized as the sustained release carrier. The biodegradable polymeric material may comprise a polylactide, a polyglycolide, a poly(lactide-co-glycolide), a polyanhydride, a polyorthoester, polycaprolactones, polyphosphazenes, polysaccharides, proteinaceous polymers, soluble derivatives of polysaccharides, soluble derivatives of proteinaceous polymers, polypeptides, polyesters, and polyorthoesters. The polysaccharides may be poly-1,4-glucans, e.g., starch glycogen, amylose, amylopectin, and mixtures thereof. The biodegradable hydrophilic or hydrophobic polymer may be a water-soluble derivative of a poly-1,4-glucan, including hydrolyzed amylopectin, hydroxyalkyl derivatives of hydrolyzed amylopectin such as hydroxyethyl starch (HES), hydroxyethyl amylose, dialdehyde starch, and the like.

In yet other preferred embodiments, sustained-release carrier comprises a synthetic or naturally occurring gum. Examples of naturally occurring gums include, e.g., the heteropolysaccharides and homopolysaccharides. An especially preferred heteropolysaccharide is xanthan gum, which is a high molecular weight (>10.sup.6) heteropolysaccharide. Other preferred heteropolysaccharides include derivatives of xanthan gum, such as deacylated xanthan gum, the carboxymethyl ether, and the propylene glycol ester. The homopolysaccharides useful in the present invention include galactomannan gums, which are polysaccharides composed solely of mannose and galactose. Preferred galactomannan gums are those which are capable of cross-linking with the heteropolysaccharide. In particular, galactomannans which have higher proportions of unsubstituted mannose regions have been found to achieve more interaction with the heteropolysaccharide when exposed to an environmental fluid. Locust bean gum, which has a higher ratio of mannose to galactose, is especially preferred as compared to other galactomannans such as guar and hydroxypropyl guar. Other natural or synthetic gums known to those skilled in the food and pharmaceutical arts are also useful as the controlled release carrier of the invention. Such gums include alginic acid derivatives, carageenan, tragacanth, acacia, karaya, guar gum, agar, acacia, galactans, mannans, and the like. Water swellable polymers may be used in addition to or instead of gums to promote sustained-release of the active agent from the final formulation. Such water swellable polymers include cellulose ethers, carboxyvinyl polymer and the like.

Optionally, the sustained-release carrier includes a release modifying agent. A release modifying agent according to the invention includes any pharmaceutically acceptable substance which may alter, e.g. prolong or increase, the release rate of the active agent form the formulation upon exposure to an aqueous environment, e.g. gastric fluid or dissolution medium. Suitable release modifying agents which may be incorporated into the matrix formulations of the present invention include, e.g., monovalent or multivalent metal cations. Preferably, the salts are inorganic salts, including e.g., alkali metal and/or alkaline earth metal sulfates, chlorides, borates, bromides, citrates, acetates, lactates, etc. In particular, these salts include, e.g., calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride. Multivalent metal cations may also be utilized. In preferred embodiments, the release modifying agents are bivalent. Particularly preferred salts are calcium sulfate and sodium chloride. Other release modifying agents include sugars, e.g. sucrose, starches, water-soluble alkylcellulose derivatives such as hydroxypropylmethylcellulose, urea, rice bran wax, and the like.

In those embodiments including a release modifying agent any effective amount may be employed (generally from about 0.1% to about 20%, by weight).

The final sustained-release oral dosage form may contain from about 1 to about 99% (by weight) of sustained release carrier. Preferably, the weight percent of the sustained release carrier ranges from about 1 to about 80%.

In certain preferred embodiments of the present invention, the sustained release carrier is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the sustained-release carrier may further include a relatively hydrophilic material, including but not limited to materials such as hydroxyalkylcelluloses such as hydroxypropylmethylcellulose and mixtures of the foregoing.

In yet another embodiment of the present invention, the sustained release carrier(s) (with or without optional release modifying agent(s)) is added into the aqueous slurry of the novel excipient, and the aqueous slurry is then dried in such a manner as to obtain agglomerated sustained release particles.

In certain embodiments of the present invention, the tablet core includes an additional dose of the same or different active ingredient in either the hydrophobic or enteric coating, or in an additional overcoating coated on the outer surface of the tablet core (without the hydrophobic or enteric coating) or as a second coating layer coated on the surface of the base coating comprising the hydrophobic or enteric coating material. This may be desired when, for example, a loading dose of a therapeutically active agent is needed to provide therapeutically effective blood levels of the active agent when the formulation is first exposed to gastric fluid. The loading dose of medicament included in the coating layer may be, e.g., from about 10% to about 40% of the total amount of medicament included in the formulation.

In addition to the lubricant of the invention, the solid dosage forms may include additional excipients and an active ingredient(s).

### Methods of Preparing Solid Dosage Forms

Solid dosage forms comprising the lubricant of the present invention may be manufactured by standard techniques known to one skilled in the art. For example, the solid dosage form may be manufactured by the wet granulation technique. In the wet granulation technique, the drug and carrier are blended using an aqueous or organic solvent, such as denatured anhydrous ethanol, as the granulation fluid. The remaining ingredients can be dissolved in a portion of the granulation fluid, such as the solvent described above, and this latter prepared wet blend is slowly added to the drug blend with continual mixing in the blender. The granulating fluid is added until a wet blend is produced, which wet mass blend is then forced through a predetermined screen and dried in a fluid bed dryer. The dried granules are then sized. Next, the lubricant of the present invention, magnesium stearate, or another suitable lubricant and other excipient materials are added to the drug granulation, and the granulation is mixed (e.g., put into milling jar sand mixed on ajar mill for 10 minutes). The composition is pressed into a layer, for example, in a Manesty® press or a Korsch LCT press. The intermediate compression typically takes place under a force of about 50-100 Newtons. Final stage compression typically takes place at a force of 3500 Newtons or greater, often 3500-5000 Newtons. The compressed cores are fed to a dry coater press, e.g., Kilian® Dry Coaterpress, and subsequently coated with the wall materials as described herein.

In addition to one or more active ingredients, additional pharmaceutically acceptable excipients (in the case of pharmaceuticals) or other additives known to those skilled in the art (for non-pharmaceutical applications) can be added to the lubricant prior to preparation of the final product.

The complete mixture, in an amount sufficient to make a uniform batch of tablets, may then subjected to tableting in a conventional production scale tableting machine at compression pressures for that machine, e.g., about 1500-10,000 lbs/sq in. The mixture should not be compressed to such a degree that there is subsequent difficulty in its hydration when exposed to gastric fluid.

### DETAILED DESCRIPTION OF THE PREFERRED EMBODIMENTS

The following examples illustrate various aspects of the present invention. They are not to be construed to limit the claims in any manner whatsoever.

### Example 1

### Rice Bran Wax Lubricant Study

The following example assesses the lubrication performance of rice bran wax as a tablet lubricant.

Rice bran wax was evaluated against Pruv® (sodium stearyl fumarate) and magnesium stearate at a concentration of 0.25% in a formulation of microcrystalline cellulose (MCC). Materials used in the study are provided in Table 1.

TABLE 1

| Ingredients | % w/w |
|---|---|
| Emcocel 90 (microcrystalline cellulose-MCC) | 99.75 |
| Lubricant: | 0.25 |
| 1) Pruv ®-sodium stearyl fumarate, or | |
| 2) Magnesium stearate, or | |
| 3) Rice bran wax | |
| total | 100.0 |

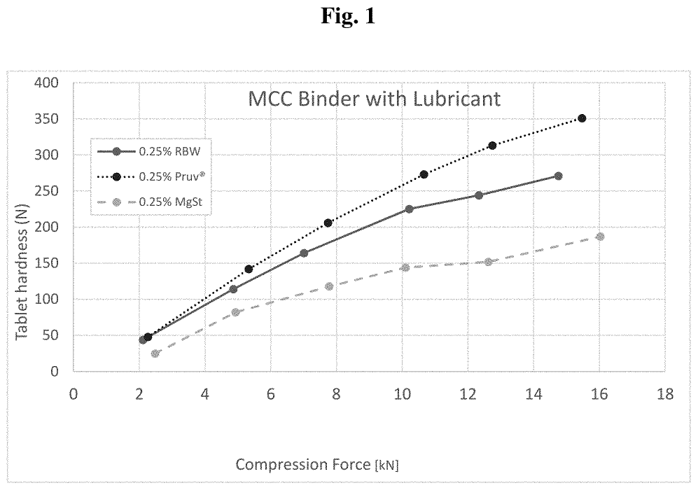

FIG. 1 is a plot of formulations containing each of the lubricants set forth in Table 1 showing tablet harness (N) at various compression forces [kN]. FIG. 1 above shows that tablets lubricated with rice bran wax produce tablets with lower hardness as compared to tablets lubricated with PRUV®, but higher hardness as compared to tablets lubricated with magnesium stearate.

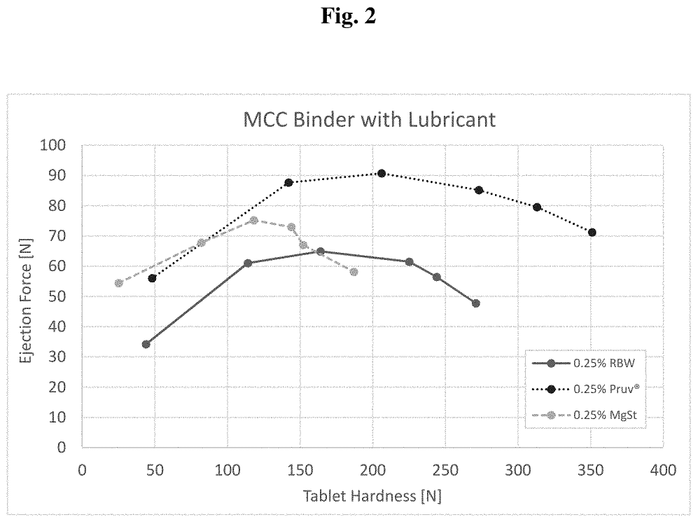

FIG. 2 is a plot of formulations containing each of the lubricants set forth in Table 1 showing ejection force [N] versus tablet harness (N). FIG. 2 shows that tablets made with rice bran wax show the lowest ejection forces of all tablets tested indicating superior lubricating efficiency.

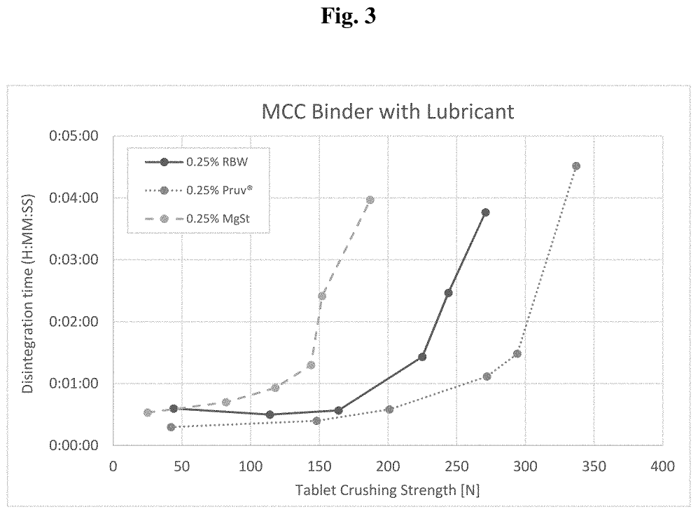

FIG. 3 is a plot of formulations containing each of the lubricants set forth in Table 1 showing disintegration time (H:MM:SS) versus tablet crushing strength [N]. FIG. 3 demonstrates that disintegration times of the tablets lubricated with rice bran wax are generally faster that those made with magnesium stearate but slightly slower than to those made with PRUV®, when compressed above 200 N.

While the tablets made with rice bran wax showed acceptable lubrication performance as compared to PRUV® and magnesium stearate overall, it was decided to seek a model that was more challenging with respect to lubrication than this microcrystalline cellulose formulation. The next set of experiments were done with dibasic calcium phosphate to provide greater discrimination of lubricant performance.

### Example 2 (Emcompress® DCP)

In Example 2, dibasic calcium phosphate (DCP) was used in this formulation example, to help differentiate the effectiveness of rice bran wax at various (four) levels (0.25%, 0.5%, 1% and 2%), and in comparison with conventionally used levels of sodium stearyl fumarate (PRUV®) and hydrogenated cottonseed oil (LUBRITAB®). Materials used in the study are provided in Table 2.

TABLE 2

| Ingredients | % w/w |
|---|---|
| Dibasic calcium phosphate (EMCOMPRESS ® DCP) | QS |
| Lubricant: | 0.25-2.0 |
| 1) Pruv ®-sodium stearyl fumarate, or | |
| 2) Lubritab ®, or | |
| 3) Rice bran wax | |
| total | 100.0 |

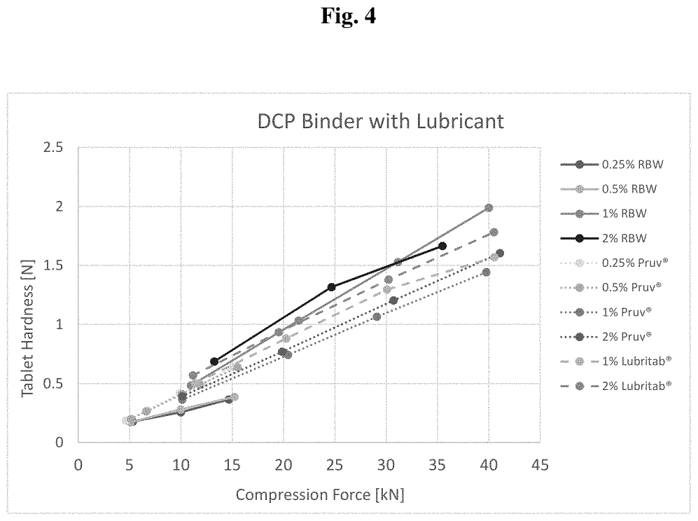

FIG. 4 is a plot of formulations containing each of the lubricants set forth in Table 2 showing tablet hardness [N] versus compression force (kN).

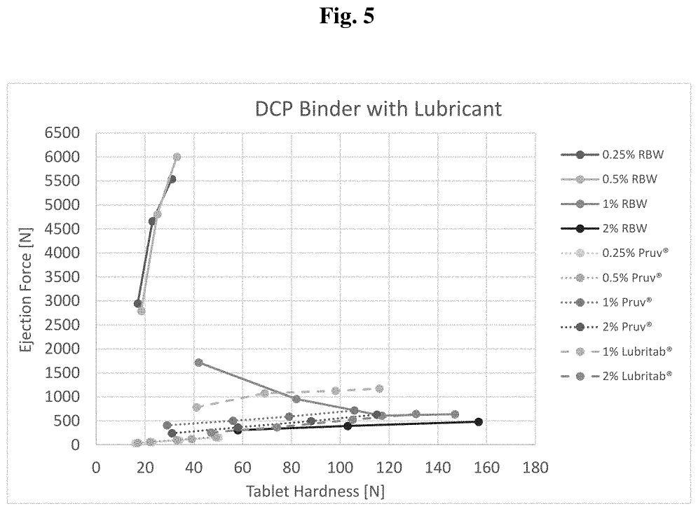

FIG. 5 is a plot of each of the lubricants set forth in Table 2 showing ejection force [N] versus tablet harness (N).

In FIG. 4, the compaction data shows that rice bran wax at low compression forces (5-15 kN) when used at 0.25-0.5%, results in tablet hardness somewhat lower than those of PRUV® when used at the same level. At compression forces above 15 kN, rice bran wax used at 0.25-0.5% did not produce viable tablets likely due to ejection stress caused by adhesion. At levels of 1-2% however, rice bran wax produced tablets of higher hardness than those produced using PRUV® and LUBRITAB® at 1-2%.

When used at 1-2%, rice bran wax, PRUV® and LUBRITAB® all produced comparable ejection forces. Note that ALL tablets in these graphs (except those made with PRUV®) showed severe sticking and picking issues, indicating that dibasic calcium phosphate is indeed a challenging model.

### Example 3 (DCP/MCC Mixtures Lubricated with Rice Bran Wax)

In Example 3, mixtures of microcrystalline cellulose and dibasic calcium phosphate were prepared and studied to find a more realistic model for further evaluation of rice bran wax. The lubricant level used in this study was 0.25%. Materials used in the study are provided in Table 3.

TABLE 3

| Ingredients | % w/w |
|---|---|
| Microcrystalline cellulose (MCC-Emcocel ® 90) | 0-100 |
| Dibasic calcium phosphate (EMCOMPRESS ® DCP) | 100-0 |
| Rice bran wax | 0.25 |
| total | 100.0 |

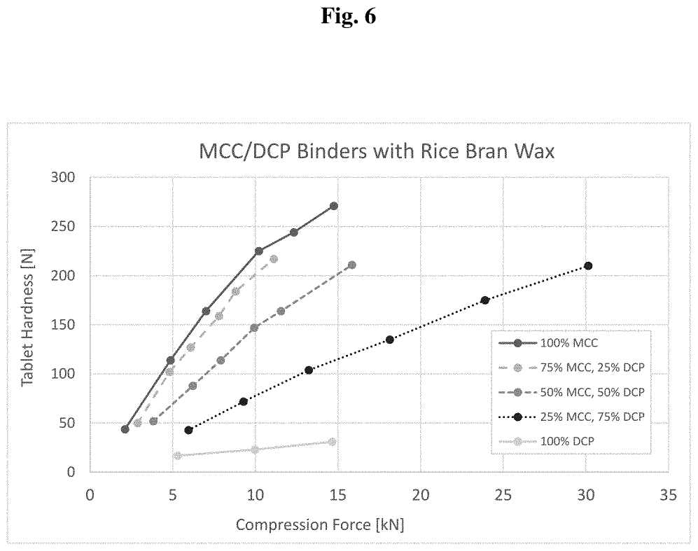

FIG. 6 is a plot of ejection force [N] versus tablet hardness [N] for formulations of Example 3 containing various amount of MCC and DCP with rice bran wax. It shows that the binding performance of dibasic calcium phosphate is much less than that of microcrystalline cellulose and increasing microcrystalline cellulose concentration from zero to 100% produces a corresponding significant increase in tablet hardness. With 50% MCC and above, good tablet hardness was achieved with 0.25% rice bran wax within a typical compression force range.

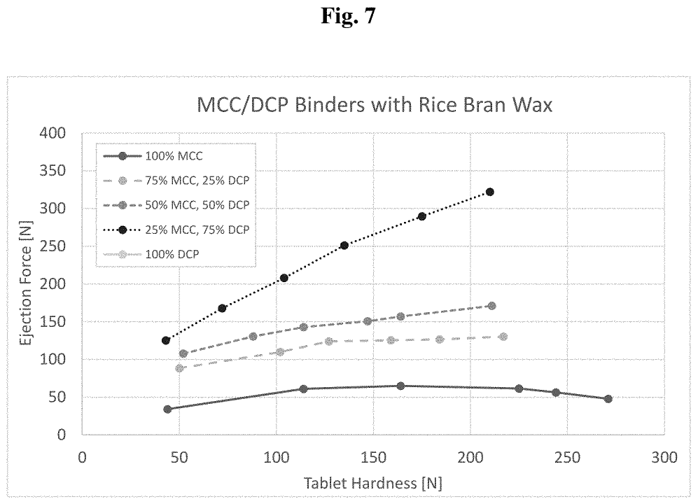

FIG. 7 is another plot of ejection force [N] versus tablet hardness [N] for formulations of Example 3 containing various amount of MCC and DCP with rice bran wax. FIG. 7 shows that the ejection forces produced in compaction of these two materials increase from a very low level for 100% MCC to a high level (off scale) with 100% DCP. This extreme overpowers the efficacy of 0.25% rice bran wax due to the abrasive nature of the DCP, also confirming the challenging nature of DCP with respect to lubrication. With 50% MCC and above, excellent ejection forces were achieved with 0.25% rice bran wax over a wide range of final tablet hardness.

### Example 4 (Evaluation of Rice Bran Wax Levels in a 1:1 DCP/MCC Mixture)

In Example 4, a 1:1 mixture of the two ingredients for further study of the lubricant at a range of concentrations from 0.1-1.0% (0.1, 0.25, 0.5 and 1%). Materials used in the study are provided in Table 4.

TABLE 4

| Ingredients | % w/w |
|---|---|
| Microcrystalline cellulose (MCC-Emcocel ® 90)/Dibasic calcium | 99.0-99.9 |
| phosphate (EMCOMPRESS ® DCP) blend 1:1 | |
| Rice bran wax | 0.1-1.0 |
| Total | 100.0 |

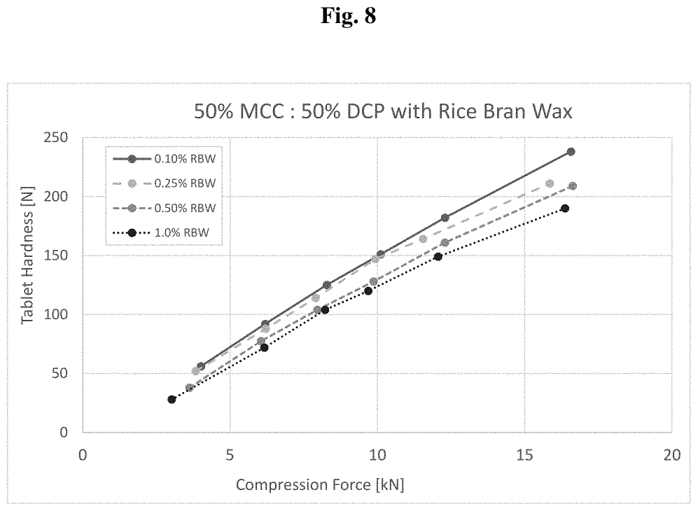

FIG. 8 is a plot of tablet hardness [N] versus compression force [kN] for formulations of Example 4 containing various amount of rice bran wax with 50% MCC:50% DCP. FIG. 8 shows that compaction of 1:1 mixtures of MCC and DCP, lubricated with four levels of rice bran wax exhibit very similar tablet hardness values indicating that the lubricant has no appreciable impact on tablet hardness across the range tested. This is notable, as tablet lubricants typically show a significant negative impact on tablet hardness as concentration is increased.

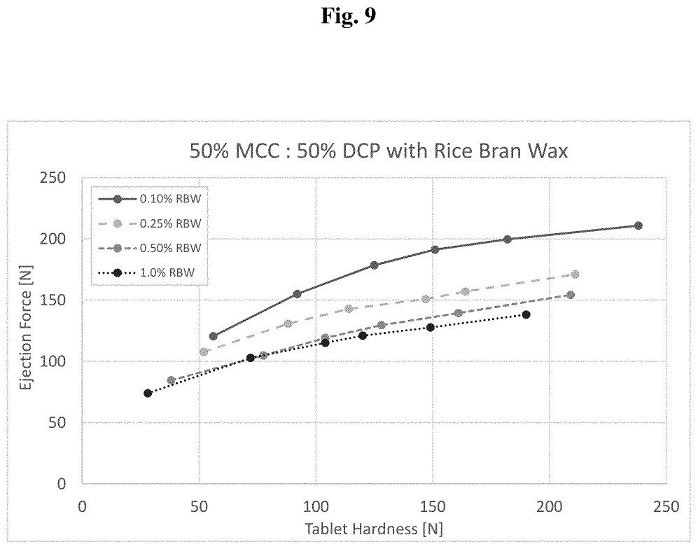

FIG. 9 is a plot showing ejection force [N] versus tablet hardness [N] for formulations of Example 4 containing 50% MCC:50% DCP with various amounts of rice bran wax (0.1, 0.25, 0.5 and 1%). Ejection forces measured across the range of lubricant concentrations (FIG. 9) show little change, indicating effective lubrication across the range of levels tested and wide range of compaction forces tested.

### Example 5 (Quercetin with 1% Rice Bran Wax in DCP/MCC)

Quercetin is a bioflavonoid polyphenol compound that has numerous anti-oxidant and general health benefits. It is a poorly water soluble crystalline powder that is challenging in flow, due to its needle shaped crystals, in compaction, and particularly in lubrication, due to its sticky nature. Several formulations were prepared as shown below with rice bran wax at 1.0%, using approximately a 1:1 ratio of MCC and DCP at two levels, and in one case the addition of a glidant (colloidal silicon dioxide), since inadequate blend flow otherwise would not allow compaction of tablets. Materials used in the study are provided in Table 5.

TABLE 5

| Formulation | 1 | 2 | 3 |
|---|---|---|---|
| Ingredients | % w/w | % w/w | % w/w |
| Quercetin dihydrate | 50.0 | 25.0 | 25.0 |
| Microcrystalline cellulose (MCC-Emcocel ® 90) | 25.0 | 37.5 | 37.5 |
| Dibasic calcium phosphate (EMCOMPRESS ® DCP) | 24.0 | 36.5 | 35.5 |
| Rice bran wax | 1.0 | 1.0 | 1.0 |
| Colloidal silicon dioxide (Cab-O-Sil ® M5P-CSD) | 0 | 0 | 1.0 |
| totals | 100.0 | 100.0 | 100.0 |

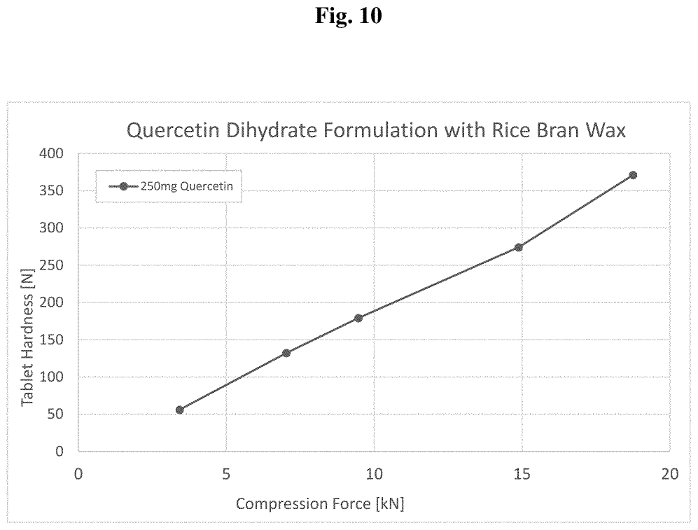

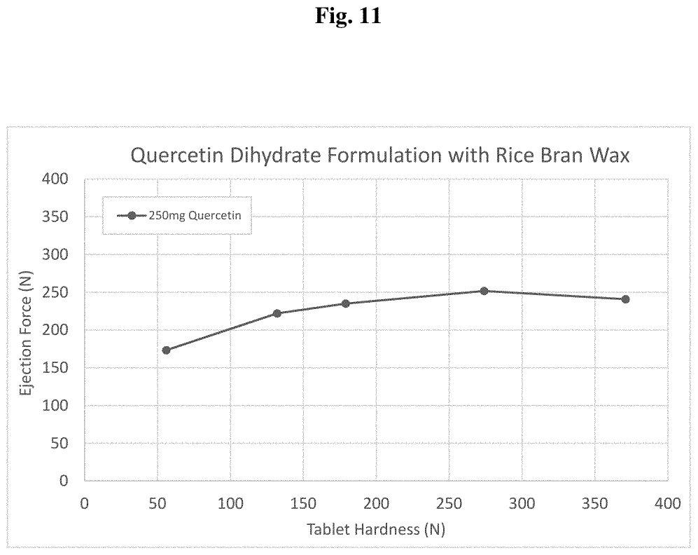

FIG. 10 is a plot showing tablet hardness [N] versus compression force [kN] for formulations containing 250 mg quercetin dehydrate and 1% rice bran wax with 50% MCC:50% DCP. Formulations 1&2 (Table 5) exhibited very poor flow and thus could not be compressed since the tablet die could not be filled, despite the use of a power feeder. Formulation 3 (Table 5), which included 1% glidant, exhibited poor but manageable flow, and with constant manual vibration of the feed hopper, tablets of satisfactory hardness were made (FIG. 10). FIG. 11 is a plot showing ejection force (N) versus tablet hardness (N) for formulations containing 250 mg quercetin dehydrate and 1% rice bran wax with 50% MCC:50% DCP and 1% glidant (Formulation #3). Formulations #1 and #2 did not contain glidant and because of this could not be compressed. Despite the sticky nature of quercetin, the 1% level of rice bran wax was sufficient to maintain low ejection forces (FIG. 11).

### Example 6 (Turmeric Formulations with Rice Bran Wax Lubricant)

Turmeric, a member of the ginger family, contains a number of terpenes including curcumin. Turmeric powder contains about 9% moisture and is very sticky in nature, making it a challenging model for tablet lubricant evaluation. In Example 6, five lubricants (rice bran wax, Pruv®, magnesium stearate, Lubritab®, and stearic acid) were compared in a 25% turmeric formulation using a 1:1 ratio of MCC and DCP. Materials used in the study are provided in Table 6.

TABLE 6

| Formulation | 1 | 2 | 3 | 4 | 5 |
|---|---|---|---|---|---|
| Ingredients | % w/w | % w/w | % w/w | % w/w | % w/w |
| Turmeric powder | 25.0 | 25.0 | 25.0 | 25.0 | 25.0 |
| Microcrystalline cellulose | 37.0 | 37.0 | 37.0 | 36.5 | 36.5 |
| (MCC-Emcocel ® 90) | | | | | |
| Dibasic calcium phosphate | 37.0 | 37.0 | 37.0 | 36.5 | 36.5 |
| (EMCOMPRESS ® DCP) | | | | | |
| Rice bran wax | 1.0 | — | — | — | — |
| Pruv ® | — | 1.0 | — | — | — |
| Magnesium stearate | — | — | 1.0 | — | — |
| Lubritab ® | — | — | — | 2.0 | — |
| Stearic acid | — | — | — | — | 2.0 |
| totals | 100.0 | 100.0 | 100.0 | 100.0 | 100.0 |

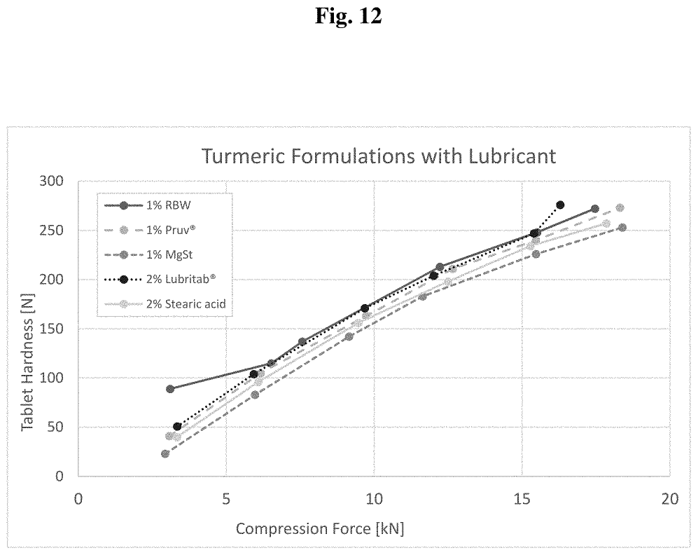

FIG. 12 is a plot of turmeric formulations with lubricant of Example 6 showing tablet hardness [N] versus compression force [kN]. With respect to compactibility (FIG. 12) all of the lubricants tested performed similarly showing no or at least a similar impact on tablet hardness.

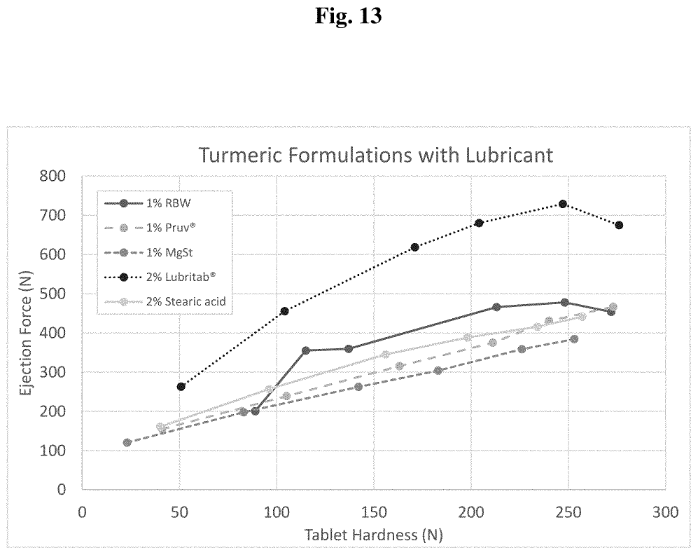

FIG. 13 is a plot of turmeric formulations of Example 6 showing ejection force (N) versus tablet hardness (N). With respect to ejection (FIG. 13) rice bran wax at 1% performed similarly to 1% Pruv® and magnesium stearate and to 2% stearic acid. Lubritab® at 2% however, exhibited higher ejection forces indicating lower lubrication efficiency.

Nearly all of the tablets made had issues with sticking to some degree. The PRUV® formulation performed the best with sticking only at compression forces of 12 and 18 kN but by comparison, rice bran wax showed significant sticking at all compaction forces tested. Magnesium stearate and Lubritab® showed sticking at 9 kN compaction and above, while stearic acid showed sticking at 6 kN compaction and above. This model formulation was clearly a challenge for punch face sticking, and the formulation can benefit from the use of an anti-adherent such as colloidal silicon dioxide or starch to reduce or eliminate this problem.

### Example 7 (Ascorbic Acid Tablets Lubricated with Rice Bran Wax Versus Stearic Acid)

Ascorbic acid (vitamin C) is a powerful anti-oxidant, and is highly hygroscopic making it prone to sticking issues in tableting. Rice bran wax was tested as a lubricant for this model formulation, in a range from 0.25% to 3% (0.25, 0.5, 1.0, 2.0 and 3.0%), and compared to stearic acid at 2%. Materials used in the study are provided in Table 7.

TABLE 7

| Formulation | 1 | 2 | 3 | 4 | 5 | 6 |
|---|---|---|---|---|---|---|
| Ingredients | % w/w | | | | | |
| Ascorbic Acid | 50.0 | 50.0 | 50.0 | 50.0 | 50.0 | 50.0 |
| ProSolv ® 90 (silicified | 45.75 | 45.5 | 45.0 | 44.0 | 43.0 | 44.0 |
| microcrystalline cellulose) | | | | | | |
| Vivasol ® (sodium starch | 4.0 | 4.0 | 4.0 | 4.0 | 4.0 | 4.0 |
| glycolate) | | | | | | |
| Rice Bran Wax | 0.25 | 0.5 | 1.0 | 2.0 | 3.0 | — |
| Stearic Acid | — | — | — | — | — | 2.0 |
| Totals | 100.0 | 100.0 | 100.0 | 100.0 | 100.0 | 100.0 |

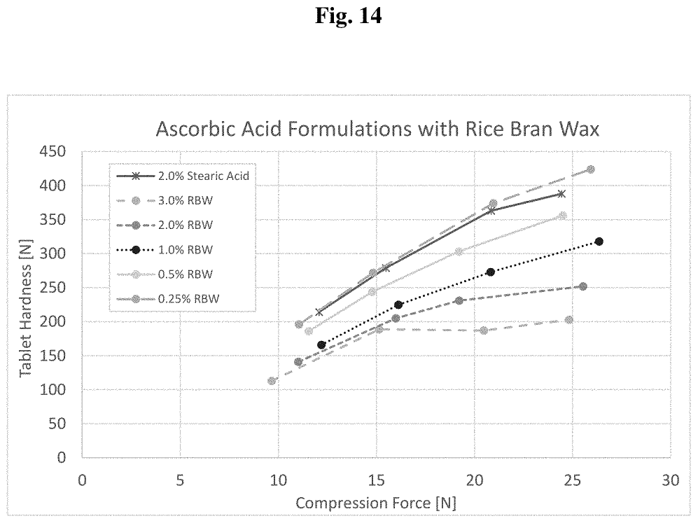

FIG. 14 is a plot of ascorbic acid formulations of Example 7 showing tablet hardness (N) versus compression force (N). The compaction plots above (FIG. 14) show a rank order performance of rice bran wax in the ascorbic acid formulation demonstrating that as rice bran wax content increased, tablet hardness decreased. This trend is generally common with most lubricants, though it was not observed in previous examples with rice bran wax. This reduction in hardness was across a fairly wide range of lubrication (0.25-3.0%), and at the highest level of rice bran wax tablets of moderate (200 N) hardness were still achievable.

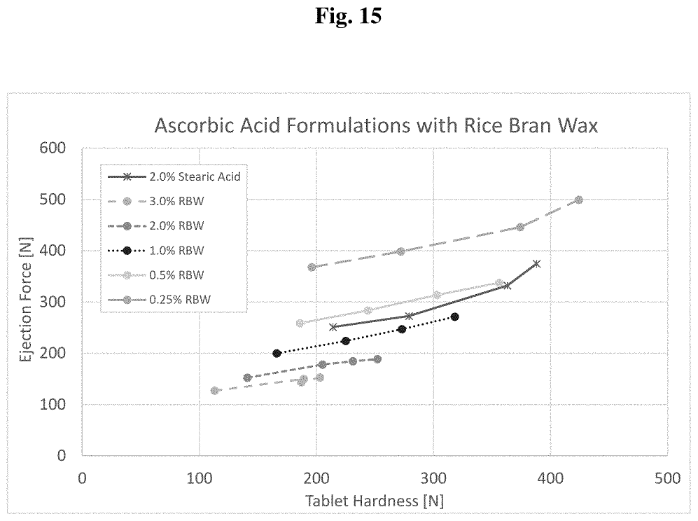

FIG. 15 is a plot of ascorbic acid formulations of Example 7 showing ejection force (N) versus tablet hardness (N). With respect to ejection force (FIG. 15) a rank-order range of results was also seen, with all but 0.25% rice bran wax producing acceptable ejection forces. It should be noted that only 0.5% rice bran wax was needed to produce an equivalent ejection force to that produced using 2.0% stearic acid (a commonly used level).

There also was some picking observed on the tablets made with 3% rice bran wax at all compaction levels and with tablets of 2% rice bran wax at the lowest compaction levels. The 2% (higher compaction level) tablets were free from picking as were all of the tablets made with 1%, 0.5% and 0.25% rice bran wax. This suggests that there may be blends in which the adhesive characteristics of rice bran wax may exceed its cohesive characteristics. In these cases, picking and sticking can be overcome by the use of an anti-adherent such as talc, starch, or silicon dioxide.

### Example 8 (Ibuprofen)

A study comparing the lubricant functionality of rice bran wax (Naturefine® R331) with that of sodium stearyl fumarate (Pruv®), magnesium stearate and hydrogenated cottonseed oil (Lubritab®) in a microcrystalline cellulose (MCC) based ibuprofen tablet model was performed. Materials used in the study are provided in Table 8.

TABLE 8

| Material | Supplier | Lot |
|---|---|---|
| Ibuprofen, USP | Spectrum | 1111254 |
| Microcrystalline | JRS | E9B19C37X |
| cellulose | | |
| Pruv ® | JRS | 2823X |
| Magnesium Stearate | Spectrum | 21G0401 |
| Lubritab ® | JRS | 1734300001X |
| Naturefine R331 | Micro Powders | CP87718 |

Five formulations, each containing a different level of lubricant, for each of the four lubricants were prepared according to Table 9. The amount of microcrystalline cellulose (MCC) was adjusted to make up for the different lubricant levels used and maintain a consistent blend size and drug concentration.

TABLE 9

| | Composition | |
|---|---|---|
| Ingredients | (% w/w) | mg/tab |
| Ibuprofen | 80.0 | 400.00 |
| Lubricant | 0.125, 0.25, 0.50, | 0.50, 1.00, 2.00, |
| | 0.75, or 1.00 | 3.00, or 4.00 |
| MCC (Emcocel ® 90M) | QS | QS |
| | 100.00 | 500.00 |

Blends consisting of five separate levels of each lubricant type were prepared, with the lubricant blending time set for up to 60 minutes. After the addition of lubricant to the ibuprofen-MCC premix and the initiation of blending, a portion of each blend (enough to make a representative number of tablets) was withdrawn at each of 5 intervals (5, 15, 30, 45 and 60 minutes). Each blend was compressed at five different compaction forces, on an instrumented tablet press, using ½ inch round, flat-face tooling.

The tablet characteristics are set forth in Table 10.

TABLE 10
Tablet Characteristics

| | |
|---|---|
| Tablet Weight | 500.0 mg |
| Tablet Shape | 0.5000” round flat faced |
| Tablet Height | 2.7-4.3 mm |

Tablet hardnesses and ejection forces were measure for each variant. When comparing tablet hardness values for the lowest levels of lubricant blended for the shortest time, tablets lubricated with sodium stearyl fumarate, magnesium stearate, hydrogenated vegetable oil and rice bran wax showed similar tablet hardness values at all tested compaction forces.

For each lubricant, at each specific compression force, when comparing tablet hardness values for the lowest levels of lubricant blended for the shortest time, with the highest levels, blended for the longest time, the following observations were made:
Tablets lubricated with Pruv®, magnesium stearate and Lubritab® all showed a drop in tablet hardness.
Tablets lubricated with Pruv® showed the least drop in tablet hardness (<3 N).
Tablets lubricated with magnesium stearate showed the highest drop in tablet hardness (between 11-20 N).
Tablets lubricated with Lubritab® showed fairly stable tablet hardness values across each of the compression forces tested.
Tablets lubricated with rice bran wax showed an increase in tablet hardness of 10-20 N with the lowest increase observed at 5 kN of compression and the highest increase at 25 kN of compression. This suggests that rice bran wax may be acting as a binder to some degree and without negatively impacting ejection forces.
When the lubricant level was increased to the highest level, tablets lubricated with magnesium stearate had lower tablet hardness values than sodium stearyl fumarate and hydrogenated vegetable oil at higher compaction forces, while rice bran wax had higher tablet hardness values at all tested compaction forces.

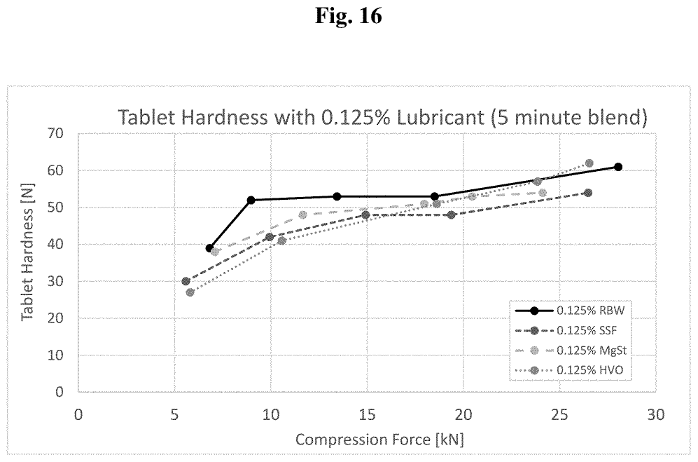

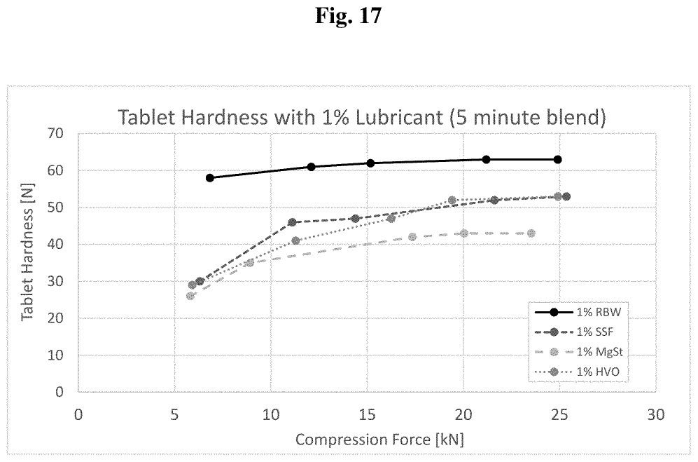

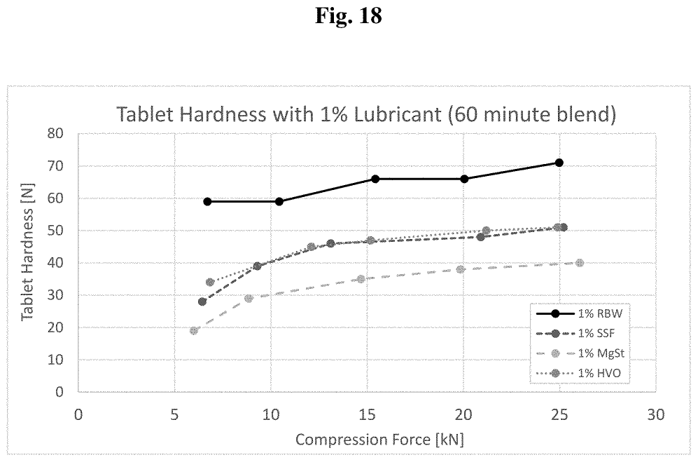

FIG. 16 is a plot that shows the tablet hardness [N] versus the compression force [kN] for the tablets of Example 8, wherein the lubricant is 0.125% rice bran wax (5 minute blend). FIG. 17 is a plot that shows the tablet hardness [N] versus compression force [kN] for the tablets of Example 8 wherein 1% lubricant (rice brain wax, sodium stearyl fumarate, magnesium stearate, hydrogenated vegetable oil; 5 minute blend) was used. FIG. 18 is a plot that shows the tablet hardness [N] versus compression force [kN] for the tablets of Example 8 wherein 1% lubricant (rice brain wax, sodium stearyl fumarate, magnesium stearate, hydrogenated vegetable oil; 60 minute blend) was used. When the blending time was also increased to the highest level, differences in tablet hardness between the lubricants became even more visible, with magnesium stearate giving the softest tablets at all tested compaction forces and rice bran wax the hardest tablets.

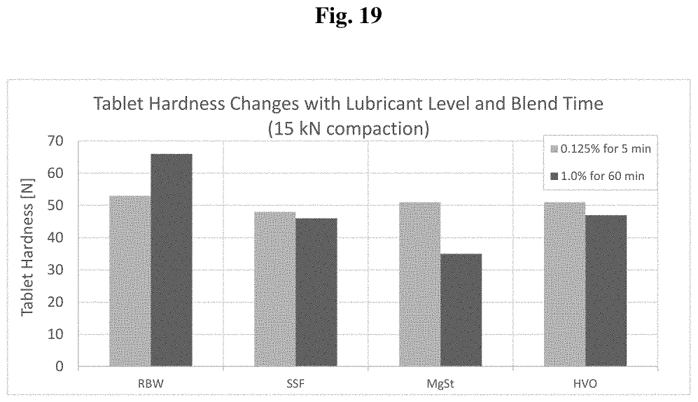

FIG. 19 is a graph which provides tablet hardness changes with lubricant level and blend time (0.125% rice bran wax for 5 minutes; 1.0% rice bran wax for 60 minutes; 15 kN compaction). When comparing tablet hardness values for the lowest levels of lubricant blended for the shortest time with the highest levels blended for the longest time, tablets lubricated with sodium stearyl fumarate, magnesium stearate and hydrogenated vegetable oil all showed a drop in tablet hardness. For example, at 15 kN compaction force, tablets lubricated with magnesium stearate showed a substantial drop in tablet hardness (˜16 N), while tablets lubricated with sodium stearyl fumarate and hydrogenated vegetable oil showed a minor drop in tablet hardness (˜2-4 N). Tablets lubricated with rice bran wax, on the other hand, showed a marked increase in tablet hardness (˜13 N).

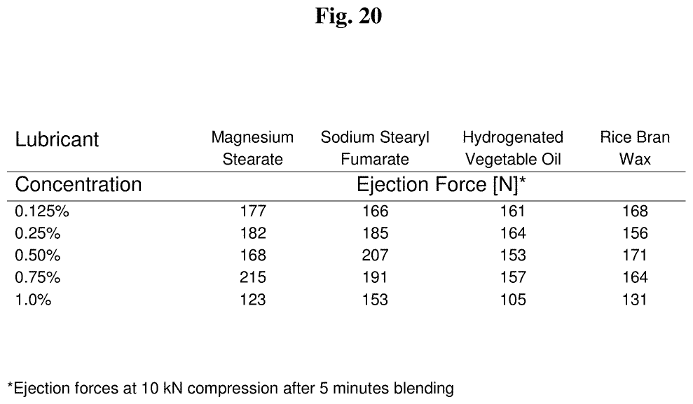

FIG. 20 is a graph which shows ejection force values for each lubricant level and 5 minute blending time. Ejection force values within each lubricant level and each blending time were generally low (100-200 N) and comparable across all four lubricants. Ejection forces for rice bran wax and magnesium stearate at 1.0% lubricant were similar after 5 minutes of blending, however the tablets formulated with rice bran wax had substantially higher tablet hardness. In fact, the tablet hardnesses were higher than with any other tested lubricant.

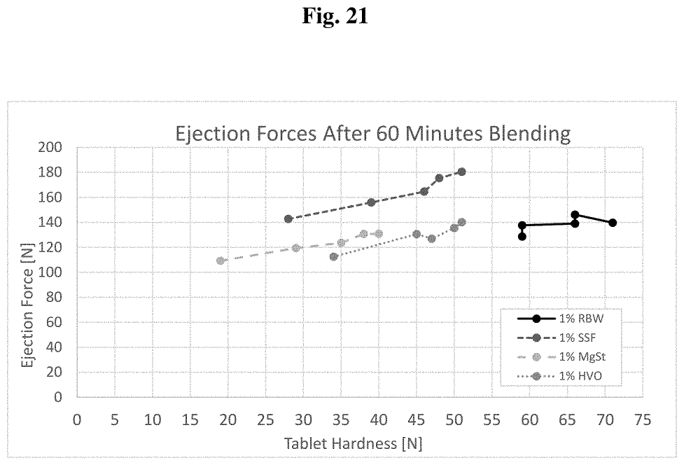

Ejection forces for rice bran wax at 1.0% lubricant slightly increased after 60 minutes of blending, while ejection forces for magnesium stearate slightly decreased. The tablet hardnesses with rice bran wax remained higher than with any other tested lubricant. FIG. 21 is a plot showing ejection forces after 60 minutes blending for 1% lubricant (rice bran wax, sodium stearyl fumarate, magnesium stearate, hydrogenated vegetable oil).

Punches were cleaned before continuing with each variant. In all cases, with all lubricants and at all conditions, some “filming” of the punches was observed. This was more severe in the blends lubricated with magnesium stearate, and picking was observed in this case.

Ejection force values within each lubricant level and each blending time were generally low (100-200 newtons) and comparable across all four lubricants.

Unlike other lubricants, increased lubricant levels and increased blending times of rice bran wax did not negatively impact tablet hardness.

### Example 9 (Rice Bran Wax/Aspirin)

Example 9 is a study which examined the lubricant-API stability of rice bran wax, in comparison with magnesium stearate. Aspirin was chosen as a model API for the study. Magnesium stearate is known to interact unfavorably with aspirin. Aspirin also has a known tendency to convert to salicylic acid in the presence of moisture.

Aspirin and lubricant were ground together at a 1:1 ratio by weight with a glass mortar and pestle, then portioned into three vials which were labeled for room temperature (ambient) storage or for storage closed or open in a stability chamber at 40° C. and 75% RH. Near-IR (NIR) spectra were obtained periodically over two months of storage under these three different conditions. Materials used in the study are provided in Table 11.

TABLE 11
Blend formulations

| | MgSt @ 1:1 | | RBW @ 1:1 | |
|---|---|---|---|---|
| Component | % w/w | g | % w/w | g |
| Aspirin | 50.0 | 8.00 | 50.0 | 8.01 |
| Magnesium | 50.0 | 8.00 | | |
| Stearate | | | | |
| Rice Bran Wax | | | 50.0 | 8.00 |
| Total | 100.0 | 16.00 | 100.0 | 16.01 |

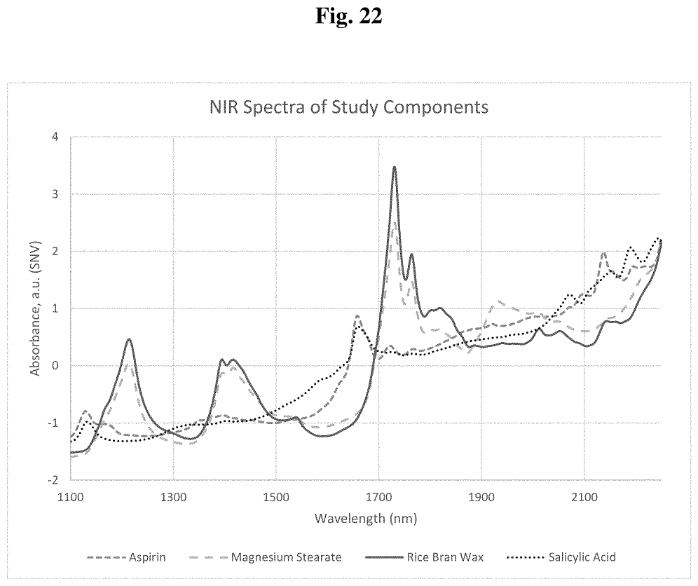

FIG. 22 is an NIR spectra of the study components of Example 9, and provides the following depictions: aspirin (close dash), magnesium stearate (far dash), rice bran wax (solid) and salicylic acid (dotted). Near IR spectra were obtained on a Metrohm 6500 RCA (Rapid Content Analyzer) reflectance NIR (JRS #1219) and a standard normal variate (SNV) pre-processing routine was used to normalize the spectra for comparison.

The NIR spectra for magnesium stearate and rice bran wax shared some common features (e.g. peaks at 1214 nm, 1394 nm, 1730 nm, 1764 nm), which were consistent with known overtone regions for alkyl chains. Some spectral differences were observed, particularly above ˜1900 nm.

The NIR spectrum for aspirin had peaks that were distinct from the lubricant peaks (e.g. peaks at 1130 nm, 1658 nm), which allowed the aspirin in the blend formulation to be monitored over time.

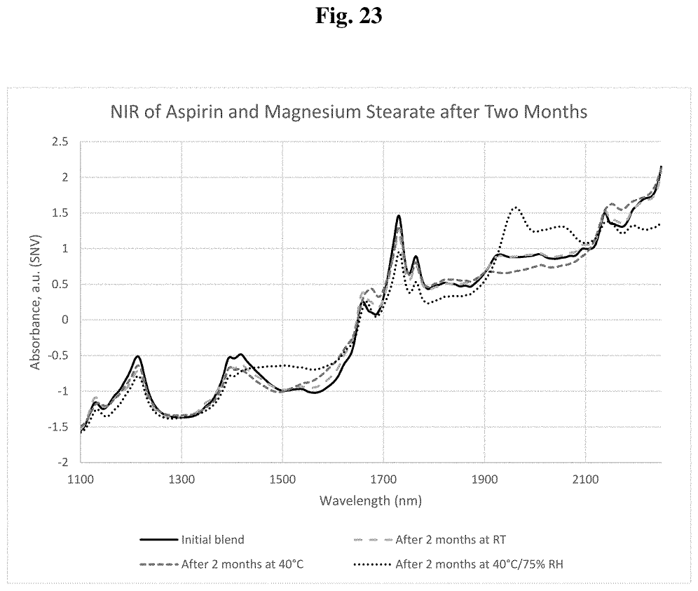

The NIR spectrum for salicylic acid showed the same two prominent peaks as the aspirin spectrum, with minor (2 nm) peak shifts (i.e. 1132 nm vs. 1130 nm and 1660 nm vs. 1658 nm). More differences were observed in the small peaks above 2000 nm. FIG. 23 is a plot showing the NIR spectra for the aspirin-magnesium stearate blend initially (solid), and after 2 months under different storage conditions (ambient—far dash, stability chamber, closed—close dash, stability chamber, open—dotted).

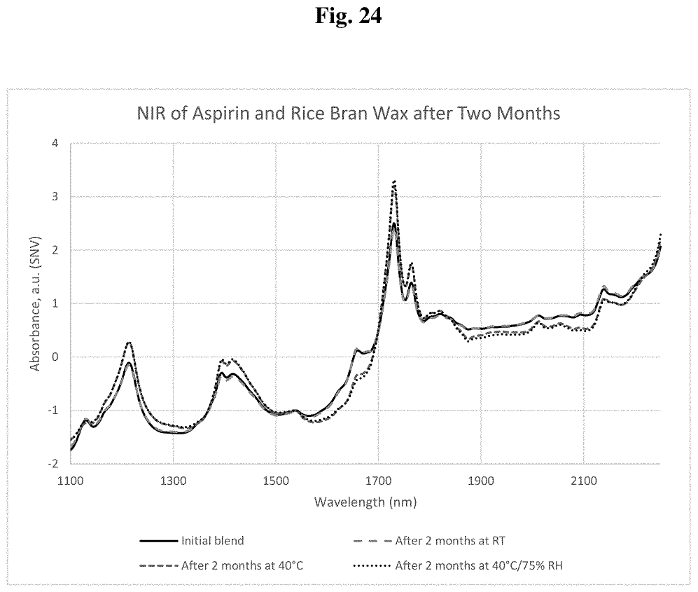

FIG. 24 is a plot showing the NIR spectra for the aspirin-magnesium stearate blend initially (solid), and after 2 months under different storage conditions (ambient—far dash, stability chamber, closed—close dash, stability chamber, open—dotted). The aspirin-magnesium stearate blend was stable to room temperature storage for two months, as shown by the close overlap of the initial (solid) and ambient (far dash) spectra in FIG. 24. The shift in the aspirin peak at 1658 nm (solid) to 1676 nm (close dash) indicates that the blend was not stable in heated storage at 40° C. The spectrum obtained after hot, humid storage open in the stability chamber at 40° C./75% RH (dotted) showed even more changes, with the aspirin peak shifting from 1658 nm to 1666 nm and broad new peaks appearing in the 1400-1600 nm region and at 1960 nm and 2050 nm. The observed shifts in the aspirin peaks may indicate Mg2+-aspirin complexation, and/or salicylic acid formation. The broad new peaks in the 1400-1600 nm and 1900-2100 nm regions are likely due to moisture inclusion.

The aspirin-rice bran wax blend was stable to room temperature storage for two months, as shown by the close overlap of the initial (solid) and ambient (far dash) spectra in FIG. 24. No shifts in the aspirin peaks were observed after heated storage at 40° C. (close dash) or hot, humid storage open in the stability chamber at 40° C./75% RH (dotted), however the peak heights diminished upon storage under these conditions.

There was no indication of interaction between rice bran wax and aspirin under any of the tested storage conditions. However, the aspirin content appeared to decline, indicating either that aspirin itself was not stable to hot and/or humid storage, or that the rice bran wax was coating more of the surface of the blend, reducing the aspirin NIR signal.

The blend of aspirin and magnesium stearate was stable to room temperature storage for 2 months. It is clear that the blend of aspirin and magnesium stearate was not stable to heated storage (i.e. in a closed vial), with additional changes introduced by exposure to high humidity during heated storage (i.e. in an open vial). The changes under heated storage (i.e. aspirin peak shift from 1658 nm to 1676 nm) are thought to be due to complexation of the Mg2+ ion from the magnesium stearate with aspirin. The difference in the aspirin peak shift under hot, humid conditions (1658 nm to 1666 nm rather than the 1676 nm seen with heated storage) is thought to be due to complexation of the Mg2+ ion from the magnesium stearate with salicylic acid rather than with aspirin, as aspirin has a known tendency to hydrolyze to salicylic acid in the presence of moisture. The additional changes under hot and humid conditions probably have to do with incorporation of moisture—while NIR spectroscopy is not well suited to explicit chemical analysis, water is known to absorb in bands around 1440-1470 nm and 1920-1940 nm, with OH stretches at higher wavelength. Overall, there were clearly signs that magnesium stearate interacts with aspirin when subjected to hot and/or humid storage.

The blend of aspirin and rice bran wax was stable to room temperature storage for 2 months. While the blend showed no signs of complexation or moisture inclusion after heated storage (i.e. in a closed vial) or heated storage with high humidity (i.e. in a open vial) for 2 months, the aspirin content in the blend appeared to decline. Overall, there was no indication of interaction between the rice bran wax lubricant and aspirin under any of the tested storage conditions, though either aspirin itself was not stable to hot and/or humid storage, or the rice bran wax was coating more of the surface of the blend, reducing the aspirin NIR signal.

### Conclusions:

The main functions of a tablet lubricant are to reduce adhesion of the tablet post compression, minimizing ejection force and punch face adhesion. An ideal tablet lubricant would achieve these functions without any negative impact on the compaction and disintegration/dissolution of the resulting tablets.

In all cases, with all lubricants at all conditions, some “filming” of the punches was observed, suggesting that the ibuprofen tablet model chosen was a challenging one. Punches were cleaned before continuing with each variant. This was more severe in the blends lubricated with magnesium stearate, and picking was observed in this case.

When compared to sodium stearyl fumarate, hydrogenated vegetable oil and magnesium stearate, rice bran wax demonstrated comparable and in some cases superior die wall lubrication (lower ejection force) and comparable reduction of punch face adhesion, in the ibuprofen tablet model studied.

Contrary to what is typical of lubricants, tablets lubricated with rice bran wax showed an increase in tablet hardness with increasing compression forces and blending times. This suggests that rice bran wax is acting as a binder to some degree (without negatively impacting ejection forces).

All of the percentages in the specification and specifically in the Examples provided above are expressed as w/w unless otherwise indicated.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A solid dosage form comprising a tableting aid which includes a lubricant, the lubricant comprising a rice bran wax, and an active ingredient.

2. The solid dosage form of claim 1, further comprising an active ingredient(s) and one or more optional pharmaceutical excipients.

3. The solid dosage form of claim 1, wherein the rice bran wax comprises from 60% to about 100% of the lubricant by weight.

4. The solid dosage form of claim 1, wherein lubricant comprises from about 0.1% to about 3% of the solid dosage form by weight.

5. The solid dosage form of claim 1, wherein the tableting aid further comprises one or more additional excipient(s).

6. The solid dosage form of claim 5, wherein the active agent comprises a drug or a nutraceutical.

7. The solid dosage form of claim 1, which is a tablet.

8. The solid dosage form of claim 6, which is a tablet.

9. A method of preparing the solid dosage form of claim 8, comprising forming a mixture comprising the active agent and the tableting aid, and compressing the mixture into a tablet.

10. A pharmaceutical tableting aid comprising rice bran wax, and at least one additional pharmaceutically acceptable excipient selected from the group consisting of an inert diluent, a binder; a disintegrant, a glidant, a flow regulator; and a decomposition accelerator.

11. The pharmaceutical tableting aid of claim 10, wherein the rice bran wax and the at least one additional pharmaceutically acceptable excipient are co-processed granules via a process selected from spray-drying, compaction or granulation.

12. The pharmaceutical tableting aid of claim 10, which comprises from about 10% to about 40% of an inert diluent; from about 0% to about 5% of a glidant; and from about 0% to about 10% of one or more additional pharmaceutically acceptable excipients.

13. The pharmaceutical tableting aid of claim 10, which includes an inert diluent.

14. The pharmaceutical tableting aid of claim 10, which includes a binder.

15. A method of preparing the pharmaceutical tableting aid of claim 10, comprising forming a mixture comprising (i) a lubricant comprising a rice bran wax, the rice bran wax comprising from about 60% to about 100% of the lubricant by weight, and (ii) at least one additional pharmaceutically acceptable excipient selected from the group consisting of an inert diluent, a binder; a disintegrant, a glidant, a flow regulator; and a decomposition accelerator, and co-processing the mixture via a process selected from spray-drying, compaction or granulation.

16. A method of preparing a compressed solid dosage form, comprising forming a mixture comprising (i) a lubricant comprising from about 60% to about 100% by weight rice bran wax, and (ii) an active ingredient, and (iii) optional additional pharmaceutical excipients, wherein the rice bran wax comprises from about 0.1% to about 3% of the solid dosage form by weight, and compressing the mixture into tablets.

17. The method of claim 15, further comprising co-processing the rice bran wax and the at least one additional pharmaceutically acceptable excipient via a process selected from spray-drying, compaction or granulation, and thereafter mixing the co-processed tableting aid with an effective amount of an active agent, and optionally compressing the mixture into tablets.

18. The solid dosage form of claim 1, wherein the active ingredient is a drug or a nutraceutical.

19. The solid dosage form of claim 1, wherein the solid dosage form comprises a compressed material, and the rice bran wax is of a type and in an amount that, upon compression into the solid dosage form, provides an increase in a hardness of the solid dosage form, as compared to the solid dosage form prepared from the same ingredients but which a different lubricant than rice bran wax and does not comprise the rice bran wax.

20. The solid dosage form of claim 1, wherein the rice bran wax comprises palmitic acid (C16), behenic acid (C22), lignoceric acid (C24), ceryl alcohol (C26), melissyl alcohol (C30), and one or more additional component(s) selected from a group consisting of free fatty acids, squalene and phospholipids.
